(19) United States
(12) Patent Application Publication
(10) Pub. No.: US 2025/0066863 A1
(43) Pub. Date: Feb. 27, 2025

(54) METHODS FOR DIAGNOSING AND/OR TREATING OTITIS MEDIA

(71) Applicant: Denver Health and Hospital Authority, Denver, CO (US)

(72) Inventors: Holly M. Frost, Denver, CO (US); Thresia Sebastian, Denver, CO (US)

(21) Appl. No.: 18/841,644

(22) PCT Filed: Feb. 24, 2023
(86) PCT No.: PCT/US2023/063218

§ 371 Date: Aug. 26, 2024

Related U.S. Application Data
(60) Provisional application No. 63/313,687, filed on Feb. 24, 2022.
(60) Provisional application No. 63/335,801, filed on Apr. 28, 2022.

(51) Int. Cl. C12Q 1/689; C12Q 1/686; G16H 50/20

(52) U.S. Cl. CPC C12Q 1/689; C12Q 1/686; G16H 50/20; C12Q 2600/16; G01N 2800/52

(57) ABSTRACT

A method for identifying the etiology of acute otitis media in a clinically diagnosed subject is provided. Also provided is a method to determine a treatment protocol in a subject clinically diagnosed with otitis media.

### CROSS-REFERENCE TO RELATED APPLICATIONS

This application claims priority to U.S. Application No. 63/313,687, filed Feb. 24, 2022, and to U.S. Application No. 63/335,801, filed Apr. 28, 2022, the contents of which are incorporated herein in their entireties.

### TECHNICAL FIELD

The subject matter described herein relates to methods to determine a treatment protocol in a subject clinically diagnosed with otitis media and to methods to diagnose and/or treat otitis media.

### BACKGROUND

Otitis media, which includes acute otitis media (AOM), chronic otitis media, otitis media with effusion, secretory otitis media, and chronic secretory otitis media as examples, is a condition affecting both adults and children. Clinically, acute otitis media is defined by an abrupt onset of middle ear effusion and inflammation, diagnosed by various methods to visualize bulging of the tympanic membrane, and often including presence of effusion in the middle ear cavity. Bacterial infection accounts for a large percentage of otitis media cases. However, viruses, as well as other microbes, may also be causative agents. Because otitis media can be caused by a virus, bacteria, or both, it is often difficult to identify the exact cause and thus the most appropriate treatment.

Additionally, concerns about the development of antimicrobial resistance have led to recommendations to withhold antibiotics, unless symptoms persist or worsen, which is sometimes referred to as “observation” or a “watchful waiting strategy,” which can prolong the symptoms. Improved approaches for diagnosing the cause of otitis media are needed, to tailor the treatment accordingly.

The foregoing examples of the related art and limitations related therewith are intended to be illustrative and not exclusive. Other limitations of the related art will become apparent to those of skill in the art upon a reading of the specification and a study of the drawings.

### SUMMARY

The following aspects and embodiments thereof described and illustrated below are meant to be exemplary and illustrative, not limiting in scope.

In one aspect, a kit including a plurality of primers specific to each of beta-lactamase producing H. influenzae, non-beta-lactamase producing H. influenzae, M. catarrhalis, and S. pneumonia is provided. The primers specific to beta-lactamase producing H. influenzae and non-beta-lactamase producing H. influenzae are capable of amplifying at least one gene that encodes beta-lactamase.

In one aspect, a method to determine a treatment regimen in a subject clinically diagnosed with otitis media is provided. The method comprises determining presence or absence of H. influenzae, S. pneumoniae, and M. catarrhalis from a sample obtained from the subject, to obtain a test result, and analyzing the test result to assess one or more of (i) spontaneous resolution rate of each detected pathogen, (ii) presence of an antibiotic resistance marker, (iii) presence of an antibiotic resistant pathogen; and/or (iv) known antibiotic resistance patterns for detected otopathogens. Based on the analyzing, a treatment regimen for the subject to treat the otitis media, if present, is provided.

In another aspect, a method to determine a treatment protocol in a subject clinically diagnosed with otitis media is provided. The method comprises analyzing a test result indicating presence or absence of negative beta-lactamase H. influenzae, positive beta-lactamase H. influenzae. M. catarrhalis, and S. pneumoniae from a biological sample from the subject to determine if S. pneumonia is present or absent, wherein
(a) if S. pneumonia is present, determining (i) if one or both of M. catarrhalis and positive beta-lactamase H. influenzae are present, wherein if one or both are present, then recommending treatment with an antibiotic with beta-lactamase activity, (ii) if negative beta-lactamase producing H. influenzae is present, then recommending treatment with an antibiotic that does not require beta lactamase activity, and/or (iii) if none of positive beta-lactamase H. influenzae, negative beta-lactamase H. influenzae, and M. catarrhalis are present then recommending treatment with an antibiotic that does not require beta lactamase activity; and
(b) if S. pneumonia is not present, recommending no antibiotic treatment.

In another aspect, a method to reduce antibiotic usage in subjects pathogenically diagnosed with otitis media, where the pathogenic diagnosis comprises a determination of presence or absence of negative beta-lactamase H. influenzae, positive beta-lactamase H. influenzae. M. catarrhalis, and S. pneumoniae is provided. The method comprises evaluating the pathogenic diagnosis to determine
(i) if S. pneumonia, negative beta-lactamase H. influenzae, and positive beta-lactamase H. influenzae are absent and M. catarrhalis is present, wherein if (i) is true, providing no antibiotic treatment;
(ii) if S. pneumonia is absent and positive beta-lactamase H. influenzae or M. catarrhalis are present, wherein if (ii) is true, providing no antibiotic treatment;
(iii) if S. pneumonia is present and M. catarrhalis and/or positive beta-lactamase H. influenzae is/are present, wherein if (iii) is true, providing antibiotic treatment with an antibiotic with beta-lactamase activity; and/or
(iv) if S. pneumonia and negative beta-lactamase H. influenzae are present, and M. catarrhalis and positive beta-lactamase H. influenzae are absent, wherein if (iv) is true, providing antibiotic treatment with an antibiotic that does not require beta-lactamase activity.

In another aspect, a method for diagnosing and/or treating otitis media, comprises obtaining a test result from an assay performed on a biological sample from a subject that reports presence or absence of negative beta-lactamase H. influenzae, positive beta-lactamase H. influenzae, M. catarrhalis, and S. pneumoniae; applying a decision algorithm to the test result to determine a treatment protocol; administering, recommending administration of, or having administered the treatment protocol; and monitoring patient response to the treatment protocol.

In an embodiment, decision algorithm comprises any one of the algorithms shown in FIGS. 1-4, or portions or combinations thereof.

In an embodiment, the decision algorithm comprises evaluating the test result to determine one or more of the following:
(i) if S. pneumonia, negative beta-lactamase H. influenzae, and positive beta-lactamase are absent and M. catarrhalis is present;
(ii) if S. pneumonia is absent and positive beta-lactamase H. influenzae and/or M. catarrhalis are present;
(ii′) if S. pneumonia, negative beta-lactamase H. influenzae, positive beta-lactamase H. influenzae, and M. catarrhalis are absent,
(iii) if S. pneumonia is present and M. catarrhalis and/or positive beta-lactamase H. influenzae is/are present;
(iv) if S. pneumonia and negative beta-lactamase H. influenzae are present, and M. catarrhalis and positive beta-lactamase H. influenzae are absent; and/or
(v) if S. pneumonia, negative beta-lactamase H. influenzae, positive beta-lactamase H. influenzae, and M. catarrhalis are absent.

In another aspect, a computer-implemented method for diagnosing otitis media is provided, where the method comprises receiving a test result from an assay performed on a biological sample from a subject, wherein the test result reports presence or absence of positive beta-lactamase H. influenzae, positive beta-lactamase H. influenzae, S. pneumoniae, and M. catarrhalis; applying a decision algorithm to the test result to determine a treatment protocol, providing the treatment protocol to the subject, a medical caregiver, clinician, or pharmacist, wherein the decision algorithm comprises any one of the algorithms shown in FIGS. 1-4, or any portion or combination thereof.

In an embodiment, the algorithm comprises determining if S. pneumonia is present or absent, wherein if S. pneumonia is present, then:
(a) determining if M. catarrhalis and/or positive beta-lactamase H. influenzae is/are present,
(b) if (a) is false, determining if negative beta-lactamase H. influenzae is present or absent;
wherein if (a) is true, providing immediate antibiotic treatment with an antibiotic with beta-lactamase activity;
wherein if (b) is true (i.e., negative beta-lactamase H. influenzae is present in a diagnostic test result), providing immediate antibiotic treatment with an antibiotic that does not require beta-lactamase activity.

In an embodiment, the algorithm further comprises determining that S. pneumonia is absent, and if true (i.e., S. pneumonia is absent in a diagnostic test result) determining one or more of (c), (d), (e), (f) and (g):
(c) if H. influenzae is present or absent;
(d) if H. influenzae is present or absent and if M. catarrhalis is present or absent;
(e) if negative beta-lactamase H. influenzae and positive beta-lactamase H. influenzae are present or absent and M. catarrhalis is present or absent;
(f) if positive beta-lactamase H. influenzae and/or M. catarrhalis is/are present or absent; and/or
(g) if negative beta-lactamase H. influenzae and/or M. catarrhalis are present and/or absent.

In an embodiment, the algorithm further comprises providing or recommending a treatment based on the determining, wherein if
(i) H. influenzae is present, determining if it is positive beta-lactamase H. influenzae that is present and determining whether M. catarrhalis is present, wherein if one or both of positive beta-lactamase H. influenzae and M. catarrhalis are present, providing or recommending a prescription for an antibiotic with beta-lactamase activity where the prescription can be filled if the subject (e.g., a patient) worsens or does not improve within a designated time frame;
(ii) H. influenzae is present, determining if it is negative beta-lactamase H. influenzae that is present and determining whether M. catarrhalis is present, wherein if one or both of negative beta-lactamase H. influenzae and M. catarrhalis are present, providing or recommending a prescription for an antibiotic that does not require beta-lactamase activity where the prescription can be filled if the subject (e.g., a patient) worsens or does not improve within a designated time frame; and/or
(iii) H. influenzae is absent and M. catarrhalis is present, providing or recommending a prescription for an antibiotic with beta-lactamase activity where the prescription can be filled if the subject (e.g., a patient) worsens or does not improve within a designated time frame.

In another aspect, a method for detecting an otopathogen in a subject is provided. The method includes collecting a nasal sample from the subject, combining a portion of the nasal sample with an extraction reagent to produce a test sample, combining a portion of the test sample with an amplification reagent to produce an amplification sample including a portion of the test sample and reagents for amplification of otopathogen nucleic acid, amplifying otopathogen nucleic acid, if present, by polymerase chain reaction (PCR) or by an isothermal amplification technique, in the amplification sample to produce an amplified sample, and detecting amplified otopathogen nucleic acid, if present, in the amplified sample.

In another aspect, a method for identifying the etiology of acute otitis media in a clinically diagnosed subject is provided. The method includes collecting a nasal sample from the subject, combining a portion of the nasal sample with an extraction reagent to produce a test sample, combining a portion of the test sample with an amplification reagent to produce an amplification sample comprising a portion of the test sample and reagents for amplification of otopathogen nucleic acid, amplifying otopathogen nucleic acid, if present, by an amplification technique in the amplification sample to produce an amplified sample, detecting amplified otopathogen nucleic acid, if present, in the amplified sample. Detection of amplified otopathogen nucleic acid in the amplified sample confirms a clinical diagnosis of acute otitis media.

In another aspect, a method for detecting an otopathogen in a subject or for identifying the etiology of acute otitis media in a clinically diagnosed subject comprises collecting a sample from the subject; optionally, combining a portion of the sample with a reagent to produce a test sample; and detecting an otopathogen, if present, in the sample.

In an embodiment, detecting otopathogen comprises detection of a single nucleic acid, a single protein, or an amplified nucleic acid. In other embodiments, detecting comprises detecting an amplicon of the otopathogen in the sample.

In another aspect, a method for treating acute otitis media in a subject is provided. The method includes obtaining a nasal sample from the subject, combining a portion of the nasal sample or the test sample with an amplification reagent to produce an amplification sample comprising a portion of the test sample and reagents for amplification of otopathogen nucleic acid, amplifying otopathogen nucleic acid, if present, in the amplification sample to produce an amplified sample, and detecting amplified otopathogen nucleic acid, if present, in the amplified sample. Upon detection of amplified otopathogen nucleic acid, acute otitis media in the subject is treated with an antibiotic. The method may also include combining a portion of the nasal sample with an extraction reagent to produce a test sample.

In another aspect, a method for treating acute otitis media in a subject is provided. The method includes obtaining a sample from the subject and detecting an otopathogen, if present, in the sample. Upon detection of an otopathogen, acute otitis media in the subject is treated with an antibiotic or is recommended for no immediate antibiotic treatment. The method may also include combining a portion of the sample with a reagent to produce a test sample.

In another aspect, a method including providing a test input to a diagnostic and treatment system, the test input including data on the presence of absence of a plurality of otopathogens, the otopathogens including a positive beta-lactamase H. influenzae, a negative beta-lactamase H. influenzae, S. pneumoniae, and M. catarrhalis in a patient sample; and generating, via a treatment model implemented using a decision algorithm, at least one primary treatment protocol is provided.

In addition to the exemplary aspects and embodiments described above, further aspects and embodiments will become apparent by reference to the drawings and by study of the following descriptions.

Additional embodiments of the present methods, diagnostic assays, kits, systems, and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

Various aspects now will be described more fully. Such aspects may, however, be embodied in many different forms and should not be construed as limited to the embodiments set forth herein; rather, these embodiments are provided so that this disclosure will be thorough and complete, and will fully convey its scope to those skilled in the art.

### BRIEF DESCRIPTION OF THE DRAWINGS

FIG. 1 depicts a decision algorithm for use in an embodiment of the methods described herein.

FIG. 2 depicts a decision algorithm for use in an embodiment of the methods described herein.

FIG. 3 depicts a decision algorithm for use in an embodiment of the methods described herein.

FIG. 4 depicts a decision algorithm for use in an embodiment of the methods described herein.

### DETAILED DESCRIPTION

### I. Definitions

For convenience, certain terms employed in the specification, examples, and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The compositions of the present disclosure can comprise, consist essentially of, or consist of, the components disclosed.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about,” particularly in reference to a given quantity, is meant to encompass deviations of plus or minus five percent.

“Antibiotic” means a compound that has the ability to destroy or inhibit microorganisms and is used to treat infectious disease. This encompasses compounds produced by microorganisms, semi-synthetic or synthetic compounds that are chemical derivatives of a compound produced by microorganisms, and semi-synthetic or synthetic compounds that act on specific biochemical pathways necessary for a cell's survival.

“Clinically” as in ‘clinically diagnosed’ intends a determination by a person, such as a medical provider or a caregiver, of otitis media based on physical presentation of signs and symptoms of otitis media, such as fever, ear pain, pulling at one or both ears, swelling and/or redness of eardrum, recent cold or respiratory infection, etc.

“Immediate” when used in the context of treating with an “immediate antibiotic” intends an antibiotic (or a prescription for an antibiotic) that is given to a subject or patient to be taken right away or as soon as practicable, or in the case of a prescription for an antibiotic, that is to be filled right away or as soon as practicable, in order to take the antibiotic right away or as soon as practicable.

“Otitis media” as used herein includes, without limitation, otitis media, acute otitis media, otitis media with effusion, secretory otitis media, and chronic secretory otitis media

“Otitis inflammation” and “ear infection” as used herein include, without limitation, otitis media and otitis externa.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in animals (e.g., mammals), and more particularly, in humans.

The term “treating” is used herein, for instance, in reference to methods of treating otitis media, and generally includes the administration of a compound or composition which reduces or ameliorates the signs and symptoms of the medical condition, or reduces the frequency of or delays the progression of a medical condition (e.g., otitis media) in a subject relative to a subject not receiving the compound or composition. This can include reversing, reducing, or arresting the symptoms, clinical signs, and/or underlying pathology of a condition in a manner to improve or stabilize a subject's condition.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications, and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications, and publications cited and this disclosure.

### II. Diagnostic Assays

Otitis media can be caused by a virus, bacteria, or both. The methods and diagnostic assays provided herein provide for identification of the cause(s) of the infection and a decision algorithm to determine a treatment regimen or protocol based on the identified cause(s).

Suitable samples (specimens) for use in the diagnostic assays disclosed herein include biological material obtained from the nose, ear, or eye of a subject (patient). A sample from the nose (nasal sample) may be a nasopharyngeal sample, an anterior naris sample, an inferior turbinate sample, a middle turbinate sample, or a superior turbinate sample. Biological material may include fluid and mucous. Fluid may be as sputum, oropharyngeal, saliva, middle or inner ear fluid, or fluid leaking from a ruptured tympanic membrane. Mucous may be nasal or conjunctival. Samples may be obtained by swabbing, wiping, or otherwise collecting biological material. Samples may be obtained indirectly, such as from surgical instruments used to place an ear tube (e.g., myringotomy tube or tympanostomy tube) or from a tube itself. When a sample obtained from other than the ear is used in the assays disclosed herein to determine the bacteria and/or virus causative of otitis media, the bacteria and/or virus in the sample is representative of the bacteria and/or virus in the ear. A sample, or a portion thereof, may be combined with an extraction reagent to produce a test sample, such as for use in an assay.

In some embodiments, samples can be analyzed for the presence of pathogens, such as otopathogens, by diagnostic assays including molecular assays. Compared to known assays and methods for diagnosing otitis media, the presently disclosed assay's may not include culturing a sample, such as to grow and then interrogate pathogens in the sample. A determination of the causative agent may be made without growing or attempting to grow any organism. Compared to known assays and methods for diagnosing otitis media, the presently disclosed assays may be free of samples obtained from the ear. The presently discloses assays may utilize samples obtained from bodily regions other than the ear, such as the nose or nasopharynx. Compared to known methods of diagnosing otitis media via a sample from the ear, obtaining a nasal or nasopharyngeal sample may be easier, less invasive, and/or less painful.

The assays disclosed herein may be as sensitive as, or more sensitive than, known otitis media assays, such as those that include culturing a sample. The sensitivity of the assays disclosed herein may be as high as, or higher than, known otitis media assays. The presently disclosed assays may take less time to run, and/or produce results faster, than known assays. For example, results from a presently disclosed assay may be available in one to several hours, and the results from culturing a sample may be available in one to several days. The assays disclosed herein may also be less expensive to run that known assays.

Diagnostic assays include, but are not limited to, assays which comprise amplification and detection of target nucleic acid sequences, such as sequences present in the genomes of pathogens and/or otopathogens. Other diagnostic assays contemplated herein do not require amplification of target pathogen nucleic acid prior to detection of such pathogenic genetic materials.

For example, in some embodiments, the diagnostic assays for otopathogen detection provided herein comprise polymerase chain reaction (PCR) based assays. PCR amplifies a specific target region of a DNA strand (the DNA target) that may be present in the samples of the present technology. If a particular otopathogen is present in a sample, then nucleic acids specific to said otopathogen can be amplified and detected with primers and probes specific for the pathogen.

As understood by one of skill in the molecular diagnostic arts, in some embodiments, a PCR-based assay comprises a polymerase, primers complementary to the DNA target (such as otopathogenic nucleic acids), and deoxynucleoside triphosphates (dNTPs) present in a suitable buffer for nucleic acid amplification and detection.

To initiate a diagnostic assay as described herein, a sample is collected and placed in suitable media to produce a test sample for storage, transport, extraction, and/or analysis. Alternatively or additionally, a sample may be combined with an extraction reagent to produce a test sample. A portion of the test sample can then be used as the basis for diagnostic analysis. The test sample, or portion thereof, may be combined with one or more reagents, such as an amplification reagent, for use in diagnostic analysis. The analysis may be by molecular assays including PCR analysis. PCR-based analyses, as described herein, include any PCR-based assays as understood by one skilled in the molecular biological arts.

For example, in some embodiments, the diagnostic assays of the present technology may include, but not be limited to, reverse transcription PCR (RT-PCR), real-time RT-PCR, isothermal PCR, multiplex PCR, and/or single cell sequencing. RT-PCR is a laboratory technique combining reverse transcription of RNA into DNA (in this context called complementary DNA or cDNA) and amplification of specific DNA targets using PCR. Multiplex polymerase chain reaction (multiplex PCR) refers to the use of polymerase chain reaction to amplify several different DNA sequences simultaneously (as if performing many separate PCR reactions all together in one reaction). Isothermal amplification methods provide detection of a nucleic acid target sequence in a streamlined, exponential manner, and are not limited by the constraint of thermal cycling. Single-cell sequencing involves isolating a single cell, amplifying the whole genome or region of interest, constructing sequencing libraries, and then applying next-generation DNA sequencing.

Accordingly, as described herein, the present technology includes utilizing the diagnostic assays described above for analysis of a sample, such as a nasal or nasopharyngeal sample, for the presence of pathogens, including, but not limited to, otopathogens, such as H. influenzae, M. catarrhalis, and/or S. pneumoniae. In some embodiments, the diagnostic assays described herein provide identification of various strains of pathogens, such as whether the pathogen expresses beta-lactamase or not.

In an embodiment, the diagnostic assay is a point-of-care assay, which allows patients, physicians, and care teams to receive test results quickly, which allows for better and more immediate clinical management decisions to be made. Point-of-care testing may be defined as medical diagnostic testing that is performed at a location where care or other treatment is provided. A point-of-care system or device may be located, for example, in a hospital, nursing home, clinic, or in the home of an individual patient. Point-of-care testing may also be referred to herein as near-patient testing, remote testing, satellite testing, and/or rapid diagnostics testing.

The present technology includes diagnostic, such as molecular diagnostic, assays for detection of pathogens from patient samples such as nasal and/or nasopharyngeal samples. In some embodiments, the diagnostic assays include PCR-based assays comprising oligonucleotides, such as primers and probes, for amplification and/or analysis and/or detection of pathogenic nucleic acids. In some embodiments the pathogens analyzed/detected by the current technology comprise otopathogens, including H. influenzae, M. catarrhalis, or S. pneumoniae. Accordingly, the current technology provides primers and probes for the analysis of otopathogens including H. influenzae, M. catarrhalis, or S. pneumoniae.

Specifically, in embodiments, the present technology is related to oligonucleotides, such as probes and primers for amplification and/or analysis of H. influenzae, such as the sequences and primers associated with H. influenzae as described in US Patent Application Publication No. 2010/0034822, incorporated by reference herein. For example, oligonucleotides, such as primers and probes, for H. influenzae may be directed to amplification and/or analysis/detection of any of the nucleic acid portions of H. influenzae provided in the odd SEQ ID NOs between 1 and 3706 of US Patent Application Publication No. 2010/0034822.

Specifically, in embodiments, the present technology is related to oligonucleotides, such as probes and primers for amplification and/or analysis of M. catarrhalis, such as the sequences and primers associated with M. catarrhalis as described in US Patent Application Publication No. 2007/0010665, incorporated by reference herein. For example, oligonucleotides, such as primers and probes, for M. catarrhalis may be directed to amplification and/or analysis/detection of any of the nucleic acid portions of S. pneumoniae provided in Table 2 of US Patent Application Publication No. 2007/0010665.

Specifically, in embodiments, the present technology is related to oligonucleotides, such as probes and primers for amplification and/or analysis of S. pneumoniae, such as the sequences and primers associated with S. pneumoniae as described in US Patent Application Publication No. 2007/0009900, incorporated by reference herein. For example, oligonucleotides, such as primers and probes, for S. pneumoniae may be directed to amplification and/or analysis/detection of any of the nucleic acid portions of S. pneumoniae provided in SEQ ID NO: 1-SEQ ID NO: 2661 or in Table 2 of US Patent Application Publication No. 2007/0009900.

A sample, as disclosed herein, may be subjected to an assay disclosed herein and the results, which may be presented in a report, may indicate the presence of absence of an otopathogen.

### A. Pathogens

The pathogenic agents, also referred to as otopathogens, can be bacterial or viral. Bacterial otopathogens include, for example, Haemophilus influenzae (H. influenzae), Moraxella catarrhalis (M. catarrhalis), Streptococcus pneumoniae (S. pneumoniae), Streptococcus pyogenes, Staphylococcus intermedius, Staphylococcus epidermidis, Staphylococcus aureus, Staphylococcus caprae, Staphylococcus auriculis, Staphylococcus capitis, Staphylococcus haemolytis, Pseudomonas aeroginosa, Proteus mirabilis, Proteus vulgaris, Escherichia faecalis, and/or Escherichia coli.

Accordingly, in an embodiment, a diagnostic assay with reagents to determine presence or absence of 1, 2, 3, 4, 5, 6, 7, 8, 9, 10, 11, 12, 13, 14, 15, 16, 17, 18, 19, 20 or more pathogenic agents is provided.

Moraxella catarrhalis (M. catarrhalis) is a Gram-negative diplococcus frequently found as a commensal of the upper respiratory tract.

Haemophilus influenzae (H. influenzae) is a Gram-negative, facultatively anaerobic coccobacillus. Depending on the presence of a polysaccharide capsule, isolates of H. influenzae are divided into encapsulated and non-encapsulated strains. There are six types of encapsulated strains designated a-f, which express distinct capsular polysaccharides, and which can be differentiated by their ability to agglutinate with antisera against the respective polysaccharide. Non-encapsulated H. influenzae strains are termed nontypable H. influenzae. Typing systems include biotyping, classification of outer membrane protein molecular weight, genetic classification by electrophoresis or PCR, and others.

Some isolates of H. influenzae are beta-lactamase positive, producing one or more beta-lactamase enzymes. Beta-lactamase may provide resistance to beta-lactam antibiotics. Other isolates of H. influenzae are beta-lactamase negative and do not produce beta-lactamase. Reference herein to a negative beta-lactamase H. influenzae intends an isolate of H. influenzae that does not or is unlikely to produce (or express) beta-lactamase. A negative beta-lactamase H. influenzae may carry a gene for beta-lactamase but not produce or express beta-lactamase. Reference herein to a positive beta-lactamase H. influenzae intends an isolate of H. influenzae that expresses (or produces) or is likely to express or produce beta-lactamase. A positive beta-lactamase H. influenzae carries a gene for beta-lactamase. A positive beta-lactamase H. influenzae isolate may be TEM-1 or ROB-1. In an embodiment, H. influenzae that does not or is unlikely to produce beta-lactamase is referred to as a “non-beta lactamase producing H. influenzae” or a negative beta-lactamase H. influenzae. In an embodiment, H. influenzae that produces beta-lactamase or is likely to produce beta-lactamase is referred to as a “beta-lactamase producing H. influenzae” or a positive beta-lactamase H. influenzae. As can be appreciated, and as described infra, a diagnostic assay can be capable of a genomic identification, to distinguish, differentiate, and/or identify with specificity a type, subtype, isolate, or strain of H. influenzae, including whether it carries a beta-lactamase gene. Other diagnostic assays can be configured to be a phenotypic indicator of H. influenzae type, subtype, isolate, or strain, and/or can be used for determining presence or absence of beta-lactamase, in order to determine whether an isolate or strain of H. influenzae in a sample is a positive beta-lactamase H. influenzae or a negative beta-lactamase H. influenzae.

In one embodiment, bacterial otopathogens are selected from gram-positive bacteria and gram-negative bacteria. The gram-positive bacteria are selected from one or more of Staphylococcus aureus, Staphylococcus epidermidis, Streptococcus agalactiae, Streptococcus pneumoniae, Streptococcus pyogenes, Enterococcus spp., and Clostridium difficile. The gram-negative bacteria are selected from one or more of Citrobacter spp., Citrobacter freundii, Enterobacter cloacae, Klebsiella pneumoniae, Escherichia coli, Proteus vulgaris, Salmonella spp., Serratia marcescens, Shigella spp., Pseudomonas aeruginosa, Moraxella mucositis, Neisseria gonorrhoeae, Neisseria meningitidis, Acinetobacter spp., Burkholderia spp., Campylobacter spp., Helicobacter pylori, Vibrio cholerae, Klebsiella pneumoniae, Haemophilus influenzae, Mycobacterium avium complex, Mycobacterium abscessus, Mycobacterium kansasii, Mycobacterium ulcerosa, Chlamydophila pneumoniae, Chlamydia trachomatis, β-hemolytic Streptococcus, Acinetobacter baumannii, Pesudomonas pyocyaneum, Bacteroides fragilis, Bacillus cereus, and Stenotrophomonas maltophilia.

In one embodiment, a bacterial otopathogen carriers one or more antimicrobial resistance markers, such as ctx-M (blaCTX-M, subgroup 1), kpc (blaKPC), mecA, ndm (blaNDM), oxa-23 (blaOXA-23), oxa-24 (blaOXA-24), oxa-48 (blaOXA-48), oxa-58 (blaOXA-58), tem (blaTEM), and vim (blaVIM).

In one embodiment, viral otopathogens are, for example, Influenza A, Influenza B. Rhinovirus, SARS-CoV-2, a rhinovirus, Middle East respiratory syndrome-coronavirus (MERS-CoV), a coronavirus, or a respiratory syncytial virus (RSV).

### B. Antibiotics

Antibiotics for managing and/or treating otitis media are now described.

Bactericidal antibiotics kill a bacteria, usually by interfering with the formation of a bacterial cell wall or its contents. A bacteriostatic antibiotic will stop a bacterial from multiplying. There are numerous different antibiotics, many of which can be classified as shown in the following groups.

Penicillins: ampicillin, amoxicillin with clavulanic acid (co-amoxiclav), dicloxacillin, flucloxacillin, nafcillin, oxacillin, penicillin, penicillin V, penicillin G, phenoxy methylpenicillin

Cephalosporins: cefaclor, cefazolin, cefadroxil, cephalexin (cefalexin), cefuroxime, cefixime, cefoxitin, ceftriaxone

Aminoglycosides: gentamicin, tobramycin

Tetracyclines: doxycycline, lymecycline, minocycline, sarecycline, tetracycline

Macrolides: azithromycin, clarithromycin, clindamycin, erythromycin, fidaxomicin, roxithromycin

Fluoroquinolones: ciprofloxacin, levofloxacin, moxifloxacin, ofloxacin; these are generally broad-spectrum antibiotics that have been used to treat a wide range of infections, especially respiratory and urinary tract infections.

Sulfonamindes: sulfamethoxazole with trimethoprim, sulfasalazine, sulfacetaminde, sulfadiazine silver.

Other antibiotics include chloramphenicol (often used for eye and ear infections), fusidic acid (often used for skin and eye infections), and nitrofurantoin and trimethoprim (often used for urinary tract infections).

Broad-spectrum antibiotics are those that act on the two major bacterial groups—gram-positive and gram-negative—or any antibiotic that acts against a wide range of disease-causing bacteria. Examples include doxycycline, minocycline, aminoglycosides (except for streptomycin), ampicillin, amoxicillin, amoxicillin/clavulanic acid (Augmentin), azithromycin, carbapenems (e.g. imipenem), piperacillin/tazobactam, quinolones (e.g., ciprofloxacin), tetracyclines, chloramphenicol, ticarcillin, and trimethoprim/sulfamethoxazole (Bactrim). Narrow-spectrum antibiotics are those that are effective against only a specific group of bacteria, such as findaxomicin and sarecycline.

Table A provides non-limiting antibiotic options for use with the methods described herein. As seen in Table A, a first treatment protocol is to provide or recommend to provide a narrow-spectrum antibiotic and/or an antibiotic that does not require beta-lactamase activity. Non-limiting examples of antibiotics for use in a treatment or treatment protocol where an antibiotic that does not require beta-lactamase activity is provided or recommended include amoxicillin, penicillin V, and phenoxymethylpenicillin. In embodiments, the narrow-spectrum antibiotic and/or an antibiotic that does not require beta-lactamase activity is selected from penicillin V, cefdinir, cefuroxime, cefpodoxime, ceftriaxone, and cefixime; 2^{nd} and 3^{rd} line cephalosporins, such as cefprozil, cefuroxime-axetil, ceftriaxone; cefdinir, cefuroxime, cefpodoxime, ceftriaxone, cefixime, co-trimoxazole, cefaclor, cefuroximexetil, clarithromycin, trimethoprim-sulfamethoxazole; and cefditoren pivoxil, cefaclor, cephalexin, ceftriaxone, cefdinir, tebipenem pivoxil, tosfloxacin, ampicillin, cefpodomixine, clindamycin, trimethoprim-sulfamethoxazole, cefuroxime, and cloxacillin.

Another treatment protocol is to provide or recommend to provide a broad-spectrum antibiotic that has beta-lactamase activity. Examples of antibiotics having or with beta-lactamase activity include amoxicillin-clavulanate, cefuroxime axetil or other 2^{nd} generation cephalosporins, and ceftriaxone. In other embodiments, the broad spectrum antibiotic or antibiotic having or with beta-lactamase activity for use in a treatment or treatment protocol where an antibiotic with or having beta-lactamase activity is provided or recommended is selected from cefdinir, cefuroxime, cefpodoxime, ceftriaxone, and cefixime; 3^{rd} generation cephalosporins (e.g. ceftriaxone); clarithromycin, erythromycin, trimethoprim-sulfamethoxazole, cefdinir, cefuroxime, cefpodoxime, ceftriaxone, cefixime, co-trimoxazole, cefaclor, cefuroximexetil, clarithromycin, trimethoprim-sulfamethoxazole, and fluoroquinolones including levofloxacin. In another embodiment, the broad spectrum antibiotic that has beta-lactamase activity is selected from amoxicillin-clavulanate, cefditoren pivoxil, ceftriaxone, cefuroxime, cefaclor, cefdinir, tebipenem pivoxil, tosfloxacin, cefpodomixine, clindamycin, trimethoprim-sulfamethoxazole, and cloxacillin.

TABLE A
Antibiotics

| Narrow-spectrum antibiotics when an | |
|---|---|
| antibiotic that does not require beta- | |
| lactamase activity is indicated or | |
| recommended | |
| (some may have beta-lactamase | Broad-spectrum antibiotics with beta-lactamase |
| activity) | activity |
| First line: Amoxicillin | First line: Amoxicillin-clavulanate |
| Alternatives: Penicillin V, Cefdinir, | Alternatives: Cefdinir, Cefuroxime, Cefpodoxime, |
| Cefuroxime, Cefpodoxime, Ceftriaxone, | Ceftriaxone, Cefixime |
| Cefixime | Further Alternatives: Clindamycin (poor |
| Further Alternatives: Clarithromycin, | H. influenzae activity), trimethoprim- |
| Clindamycin (poor H. influenzae | sulfamethoxazole (high S. pneumoniae resistance), |
| activity), trimethoprim- | erythromycin-sulfisoxazole (high S. pneumoniae |
| sulfamethoxazole (high S. pneumoniae | resistance), azithromycin (high S. pneumoniae |
| resistance), erythromycin-sulfisoxazole | resistance), Linezolid, Fluoroquinolones including |
| (high S. pneumoniae resistance), | levofloxacin, combination cefixime + clindamycin |
| azithromycin (high S. pneumoniae | or ceftriaxone + clindamycin, erythromycin (high |
| resistance), erythromycin (high | S. pneumoniae resistance), doxycycline |
| S. pneumoniae resistance), doxycycline | |
| First-line: Amoxicillin | First-line: Amoxicillin-clavulanate or |
| Alternatives: 2^{nd} or 3^{rd} line | cefuroxime axetil or other 2^{nd} generation |
| cephalosporins (e.g. cefprozil, | cephalosporin |
| cefuroxime-axetil, ceftriaxone, etc.) | Alternatives: 3^{rd} generation cephalosporins (e.g. |
| Further Alternatives: Clarithromycin, | ceftriaxone) |
| azithromycin, clindamycin, | Further Alternatives: Clarithromycin, |
| doxycycline, fluoroquinolone including | azithromycin, clindamycin, doxycycline, |
| levofloxacin | fluoroquinolone including levofloxacin |
| First line: Amoxicillin or Penicillin V | First line: Amoxicillin-clavulanate or |
| or Phenoxymethylpenicillin | Ceftriaxone |
| Alternative: Cefdinir, Cefuroxime, | Alternative: Clarithromycin, erythromycin, |
| Cefpodoxime, Ceftriaxone, Cefixime, | trimethoprim-sulfamethoxazole, Cefdinir, |
| Co-trimoxazole, Cefaclor, | Cefuroxime, Cefpodoxime, Ceftriaxone, Cefixime, |
| cefuroximexetil, Clarithromycin, | Co-trimoxazole, Cefaclor, Cefuroximexetil, |
| trimethoprim-sulfamethoxazole | Clarithromycin, trimethoprim-sulfamethoxazole, |
| Further Alternatives: azithromycin, | Fluoroquinolones including levofloxacin |
| erythromycin-sulfisoxazole, | Further Alternatives: Clindamycin (poor |
| erythromycin | H. influenzae activity), erythromycin-sulfisoxazole |
| | (high S. pneumoniae resistance), azithromycin |
| | (high S. pneumoniae resistance), Linezolid, |
| | combination 2^{nd} or 3^{rd} generation cephalosporin + |
| | clindamycin or clarithromycin |
| First-line: Amoxicillin, Penicillin V | First-line (based on availability): amoxicillin- |
| Alternative: cefditoren pivoxil, | clavulanate, cefditoren pivoxil, ceftriaxone, |
| Cefaclor, cephalexin, ceftriaxone, | cefuroxime, Cefaclor, Cefdinir, tebipenem pivoxil, |
| Cefdinir, tebipenem pivoxil, | tosfloxacin, cefpodomixine, clindamycin, |
| tosfloxacin, ampicillin, cefpodomixine, | trimethoprim-sulfamethoxazole, cloxacillin |
| clindamycin, trimethoprim- | (see above for other options) |
| sulfamethoxazole, cefuroxime, | |
| cloxacillin (see above for other options) | |

The antibiotic can be provided or administered in a liquid or solid form. Tablets, capsules, a liquid, a suspension, a dispersion, a spray, and drops are contemplated. Ear drops may be used as the vehicle for administration of the antibiotic. In the case that ear swelling has progressed substantially and ear drops do not penetrate significantly into the ear canal, a wick can be inserted into the ear canal to facilitate penetration of the treatment solutions. It is also contemplated to provide a therapeutic agent other than an antibiotic, such as a pain relief medication or an antiviral agent.

Antibiotics with β-lactamase activity can include penicillins, cephalosporins, monobactams, carbapenems, and penemase inhibitors. Specifically, the β-lactam antibiotics include penicillin, oxacillin, cloxacillin, dicloxacillin, flucloxacillin, ampicillin, pivampicillin, amoxicillin, carbenicillin, furbenicillin, sulbenicillin, ticarcillin, piperacillin, mecillinam, cephalothin, cephaloridine, cefazolin, cefradine, cefuroxime, cefaclor, cefotaxime, ceftriaxone, ceftazidime, cefoperazone, cefoxitin, imipenem, aztreonam, and the like.

Treatment options include antibiotics or surgical intervention, including myringotomy, an operation to insert a tympanostomy tube through the tympanic membrane and into the patient's middle ear to drain the fluid and balance the pressure between the outer and middle ear. Antipyretics and analgesics, including benzocaine, ibuprofen, and acetaminophen, may also be prescribed to treat accompanying fever or pain symptoms. Antivirals can be administered if the diagnostic test result indicates presence of a virus.

### III. Algorithms for Analysis of Diagnostic Assay Test Result

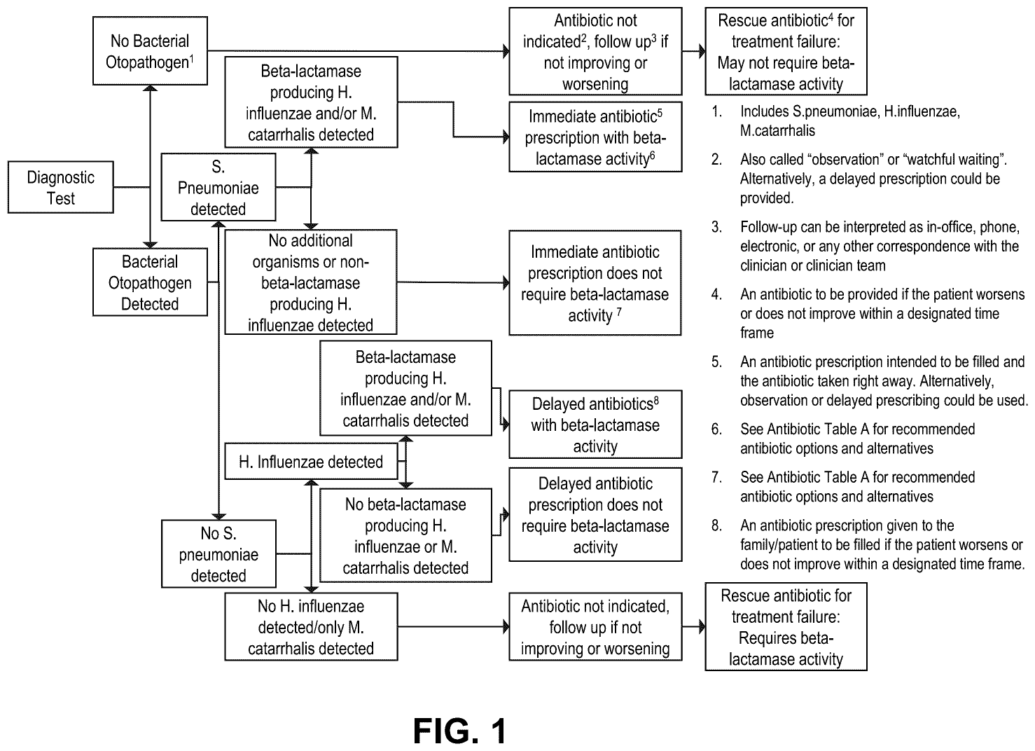

FIGS. 1-4 show embodiments of decision algorithms for evaluating or analyzing a diagnostic test result. The algorithms are used in conjunction with methods to, for example, determine a treatment regimen or treatment protocol in a subject clinically diagnosed with otitis media and/or to provide a recommendation for a treatment regimen or treatment protocol in a subject clinically diagnosed with otitis media, where the clinical diagnosis may be supplemented with a diagnostic test result or report from a diagnostic assay on a sample taken from the subject with the clinical diagnosis to determine presence or absence of one or more otopathogens. The algorithms are also used in conjunction with a diagnostic report or result from a diagnostic assay on a sample from a subject for one or more otopathogens to treat and/or recommend a treatment protocol based on presence (or absence) of certain otopathogens. The algorithms, in other embodiments, are used in conjunction with a pathogenic diagnosis of one or more otopathogens associated with otitis media, to provide a method to reduce antibiotic usage in subjects pathogenically diagnosed with otitis media.

FIG. 1 shows a first exemplary decision algorithm. The decision algorithm initiates with inspection of results or a report from a diagnostic assay performed on a sample from a subject. In some embodiments, the subject has a clinical diagnosis of otitis media. In other embodiments, the subject is one suspected of or at risk of otitis media. A sample from the subject is tested on a diagnostic assay that determines whether certain otopathogens are present in the sample. Suitable diagnostic assays, samples, and otopathogens are described above. The decision algorithm determines whether S. pneumonia is present or absent in the test result. If S. pneumonia is present, it is then determined (i) if one or both of M. catarrhalis and positive beta-lactamase H. influenzae are present, wherein if one or both are present, then treatment with an antibiotic with beta-lactamase activity is provided or recommended, (ii) if negative beta-lactamase producing H. influenzae is present, then treatment with an antibiotic that does not require beta lactamase activity is provided or recommended, and/or (iii) if none of positive beta-lactamase H. influenzae, negative beta-lactamase H. influenzae, and M. catarrhalis are present then treatment with an antibiotic that does not require beta-lactamase activity is provided or recommended. In an embodiment, the antibiotic provided or recommended is an “immediate” antibiotic, intending that the antibiotic is prescribed with instructions to fill the prescription at a pharmacy and/or to take the antibiotic as soon as practicable.

Continuing with the FIG. 1 algorithm, if S. pneumonia is not present in the diagnostic test result or report, then, in an embodiment, no antibiotic treatment is provided or recommended. In another embodiment, if S. pneumonia is not present in the diagnostic test result or report, then the algorithm determines whether H. influenzae is present or absent. If no H. influenzae is present and/or M. catarrhalis is present, then a recommendation to not provide an antibiotic is made or no antibiotic is provided. In an embodiment, where a recommendation to not provide an antibiotic is made or no antibiotic is provided, the algorithm can recommend that a follow-up be made, where the follow-up can be clinician or subject initiated, and can be in-office, in-person, via phone, via computer, electronic (e.g., via text), or any other correspondence between the subject and the clinician or clinician team. If H. influenzae is present, the diagnostic test result or report is further evaluated to determine the type of H. influenzae that is present. If positive beta-lactamase H. influenzae is present and/or M. catarrhalis is present in the test result or report, then a recommendation for a delayed antibiotic with beta-lactamase activity is made. If positive beta-lactamase H. influenzae is not present and M. catarrhalis is not present in the test result or report, then a recommendation for a delayed antibiotic that does not require beta-lactamase activity is made. A delayed antibiotic recommendation intends a prescription that is given to the subject or to a guardian, family member, friend, etc., with instructions that the prescription be filled at a pharmacy (or via mail) if the subject worsens or does not improve within a designated time frame. Exemplary designated time frames are detailed infra.

In an embodiment, for subjects with no S. pneumonia nor H. influenzae (of either subtype), and with M. catarrhalis present in the diagnostic test result or report, and where an antibiotic was not recommended or provided, with or without a recommendation for a follow-up, if the subject does not improve or worsens in a certain time frame, then a rescue antibiotic is provided or recommended, where the rescue antibiotic is one having beta-lactamase activity.

Accordingly, provided is a method to reduce antibiotic usage in subjects pathogenically diagnosed with otitis media, where the pathogenic diagnosis includes a determination of presence or absence of negative beta-lactamase H. influenzae, positive beta-lactamase H. influenzae, M. catarrhalis, and S. pneumoniae. The method includes evaluating the pathogenic diagnosis to determine the following:
(i) if S. pneumonia, negative beta-lactamase H. influenzae, and positive beta-lactamase H. influenzae are absent and M. catarrhalis is present, wherein if (i) is true (i.e., S. pneumonia, negative beta-lactamase H. influenzae, and positive beta-lactamase H. influenzae are absent and M. catarrhalis is present), providing no antibiotic treatment;
(ii) if S. pneumonia is absent and positive beta-lactamase H. influenzae or M. catarrhalis are present, wherein if (ii) is true (i.e., S. pneumonia is absent and positive beta-lactamase H. influenzae or M. catarrhalis are present), providing no antibiotic treatment;
(iii) if S. pneumonia is present and M. catarrhalis and/or positive beta-lactamase H. influenzae is/are present, wherein if (iii) is true (i.e., S. pneumonia is present and M. catarrhalis and/or positive beta-lactamase H. influenzae is/are present), providing antibiotic treatment with an antibiotic with beta-lactamase activity; and/or
(iv) if S. pneumonia and negative beta-lactamase H. influenzae are present, and M. catarrhalis and positive beta-lactamase H. influenzae are absent, wherein if (iv) is true (i.e., S. pneumonia and negative beta-lactamase H. influenzae are present, and M. catarrhalis and positive beta-lactamase H. influenzae are absent) providing antibiotic treatment with an antibiotic that does not require beta-lactamase activity.

In another aspect, a method for diagnosing and/or treating otitis media includes obtaining a test result from an assay performed on a biological sample from a subject that reports presence or absence of negative beta-lactamase H. influenzae, positive beta-lactamase H. influenzae, M. catarrhalis, and S. pneumoniae; applying a decision algorithm to the test result to determine a treatment protocol; administering, recommending administration of, or having administered the treatment protocol; and monitoring patient response to the treatment protocol.

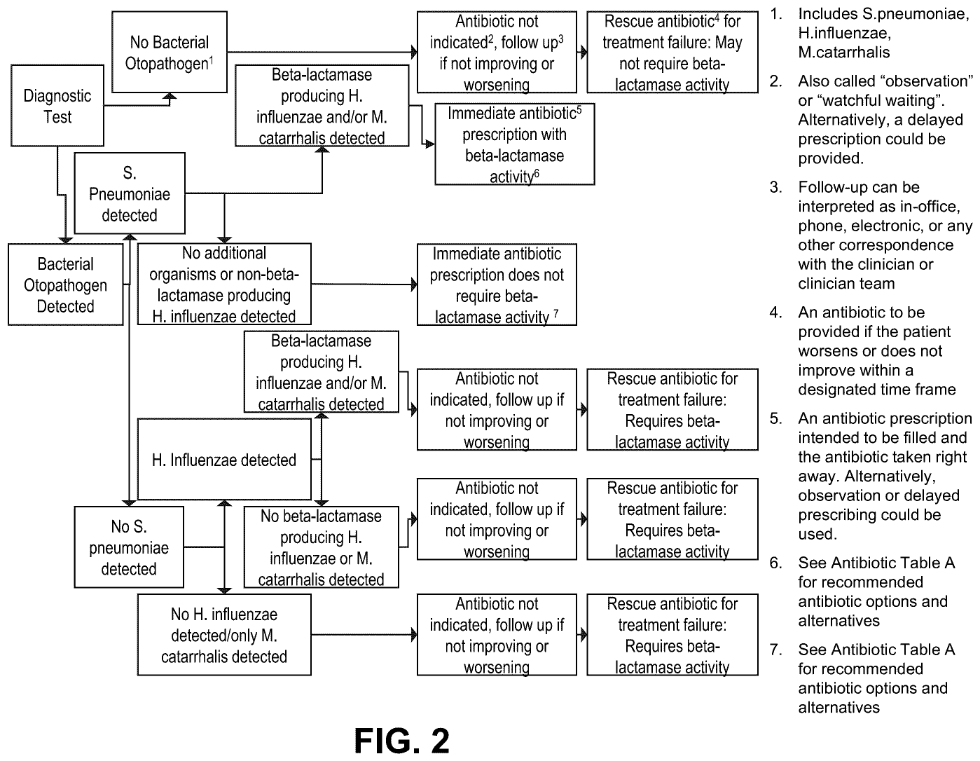

FIG. 2 depicts another exemplary algorithm for use in the methods described herein. A diagnostic test result or report is analyzed, obtained, or evaluated to determine if a bacterial otopathogen is present. If not, then no antibiotic is provided or recommended, optionally with a recommendation for or instructions to follow-up if the subject does not improve or worsens in a designated time frame. If an otopathogen is present, the result or report is analyzed or inspected to determine whether S. pneumonia is present. If it is not, then the result or report is further analyzed or inspected to determine whether any type or isolate of H. influenzae is present. If no type or isolate of H. influenzae is present, the result or report is further analyzed or inspected to determine whether M. catarrhalis is present. If M. catarrhalis is present in the report or result, but no type or isolate of H. influenzae is present, then no antibiotic is provided or recommended, optionally with a follow-up if the subject does not improve or worsens in a designated time frame. Optionally, if the subject in the absence of an antibiotic does not improve or worsens in a certain time frame, then a rescue antibiotic can be provided or recommended, where the rescue antibiotic is one having beta-lactamase activity.

If a type or isolate of H. influenzae is present in the test report or result, then the result or report is further analyzed or inspected to determine the type or isolate of H. influenzae present. In an embodiment, the report or result provides information on presence or absence of positive beta-lactamase H. influenzae. If positive beta-lactamase H. influenzae is present (and, recall, at this point in the algorithm, a determination has been made that S. pneumonia is not present), the report or result is consulted to determine whether M. catarrhalis is present. If positive beta-lactamase H. influenzae and/or M. catarrhalis is/are present, then an antibiotic is not provided or recommended, optionally with a recommendation for or instructions to follow-up if the subject does not improve or worsens in a designated time frame. Optionally, if the subject in the absence of an antibiotic does not improve or worsens in a certain time frame, then a rescue antibiotic may be provided or recommended, where the rescue antibiotic is one having beta-lactamase activity.

With continued reference to FIG. 2, if positive beta-lactamase H. influenzae is not present and negative beta-lactamase H. influenzae and/or M. catarrhalis is/are present (and, recall, at this point in the algorithm, a determination has been made that S. pneumonia is not present), then an antibiotic is not provided or recommended, optionally with a recommendation for or instructions to follow-up if the subject does not improve or worsens in a designated time frame. Optionally, if the subject in the absence of an antibiotic does not improve or worsens in a certain time frame, then a rescue antibiotic can be provided or recommended, where the rescue antibiotic is one having beta lactamase activity.

In an embodiment, the methods herein apply a decision algorithm as depicted in any one FIGS. 1-4, or any portions or any combinations thereof.

In an embodiment, the decision algorithm comprises evaluating a test result or report to determine one or more of the following:
(i) if S. pneumonia, negative beta-lactamase H. influenzae, and positive beta-lactamase H. influenzae are absent and M. catarrhalis is present;
(ii) if S. pneumonia is absent and positive beta-lactamase H. influenzae and/or M. catarrhalis are present;
(ii′) if S. pneumonia, negative beta-lactamase H. influenzae, positive beta-lactamase H. influenzae, and M. catarrhalis are absent,
(iii) if S. pneumonia is present and M. catarrhalis and/or positive beta-lactamase H. influenzae is/are present;
(iv) if S. pneumonia and negative beta-lactamase H. influenzae are present, and M. catarrhalis and positive beta-lactamase H. influenzae are absent; and/or
(v) if S. pneumonia, negative beta-lactamase H. influenzae, positive beta-lactamase H. influenzae, and M. catarrhalis are absent.
   Optionally, the algorithm provides a treatment or a treatment protocol to the subject or to the clinician based on whether (i), (ii), (ii′), (iii), (iv), and/or (v) is/are true or false (e.g., presence or absence of the otopathogens).

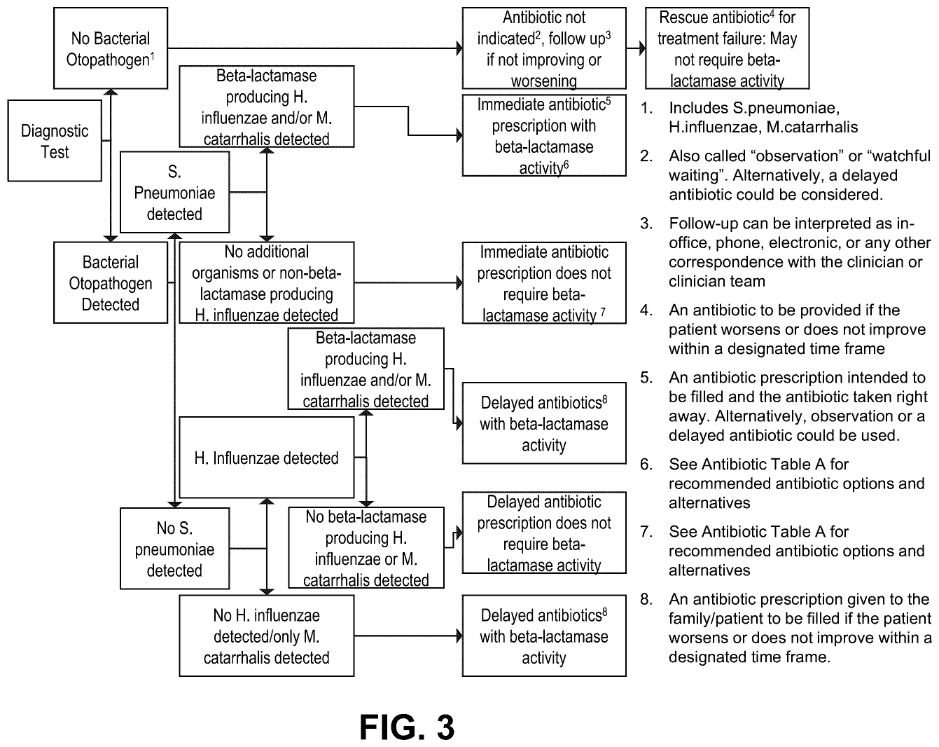

FIG. 3 depicts a decision algorithm according to another embodiment. The algorithm comprises determining from a diagnostic test result that includes or reports presence and/or absence of S. pneumonia, H. influenzae, and M. catarrhalis, such as from a diagnostic test that reports presence or absence of S. pneumonia, positive beta-lactamase H. influenzae, negative beta-lactamase H. influenzae, and M. catarrhalis, if S. pneumonia is present or absent. If S. pneumonia is present, then the method includes determining if M. catarrhalis and/or positive beta-lactamase H. influenzae is/are present; if one or both of M. catarrhalis and/or positive beta-lactamase H. influenzae is/are present, then providing or recommending to provide antibiotic treatment with an antibiotic with beta-lactamase activity. Such providing or recommending to provide treatment is referred to in the industry, and herein, as “immediate” antibiotic treatment. It will be appreciated that “immediate” intends treatment as soon as practicable for a given subject.

If S. pneumonia is present and negative beta-lactamase H. influenzae is absent and/or no other organisms (such as M. catarrhalis and positive beta-lactamase H. influenzae) are present, then the method comprises providing or recommending to provide immediate antibiotic treatment with an antibiotic that does not require beta-lactamase activity.

With continued reference to FIG. 3, if S. pneumonia is absent in the diagnostic test result, then the method includes determining whether H. influenzae is present or absent, and may include whether either of negative beta-lactamase H. influenzae and positive beta-lactamase H. influenzae are present or absent. If H. influenzae is absent, or alternatively if negative beta-lactamase H. influenzae and positive beta-lactamase H. influenzae are both absent, the method includes determining if M. catarrhalis is present or absent. If M. catarrhalis is present, then the method includes providing or recommending to provide delayed antibiotic treatment with an antibiotic with beta-lactamase activity. “Delayed” antibiotic treatment intends that a prescription for the antibiotic be provided to the subject (e.g. the patient) or to a family member, guardian, or the like of the subject, with instructions that the prescription be filled at a pharmacy if the subject worsens or does not improve within a certain time frame.

If inspection of the diagnostic test result/report reveals that S. pneumonia is absent and H. influenzae is present, then a determination of whether the H. influenzae is negative beta-lactamase H. influenzae or positive beta-lactamase H. influenzae and whether M. catarrhalis is present or absent is conducted. If positive beta-lactamase H. influenzae and/or M. catarrhalis is/are present then the method comprises providing or recommending to provide delayed antibiotic treatment with an antibiotic with beta-lactamase activity. If neither positive beta-lactamase H. influenzae nor M. catarrhalis is/are present, then the method comprises providing or recommending to provide delayed antibiotic treatment with an antibiotic that does not require beta-lactamase activity.

In an embodiment, the algorithm includes providing or recommending a treatment option from the following options, which apply if S. pneumoniae is not present and:
(i) H. influenzae is present, determining if it is positive beta-lactamase H. influenzae that is present and determining whether M. catarrhalis is present, wherein if one or both of positive beta-lactamase H. influenzae and M. catarrhalis are present, providing or recommending a prescription for an antibiotic with beta-lactamase activity where the prescription can be filled if the subject (e.g., a patient) worsens or does not improve within a designated time frame;
(ii) H. influenzae is present, determining if it is negative beta-lactamase H. influenzae that is present and determining whether M. catarrhalis is present, wherein if one or both of negative beta-lactamase H. influenzae and M. catarrhalis are present, providing or recommending a prescription for an antibiotic that does not require beta-lactamase activity where the prescription can be filled if the subject (e.g., a patient) worsens or does not improve within a designated time frame; and/or
(iii) H. influenzae is absent and M. catarrhalis is present, providing or recommending a prescription for an antibiotic with beta-lactamase activity where the prescription can be filled if the subject (e.g., a patient) worsens or does not improve within a designated time frame.

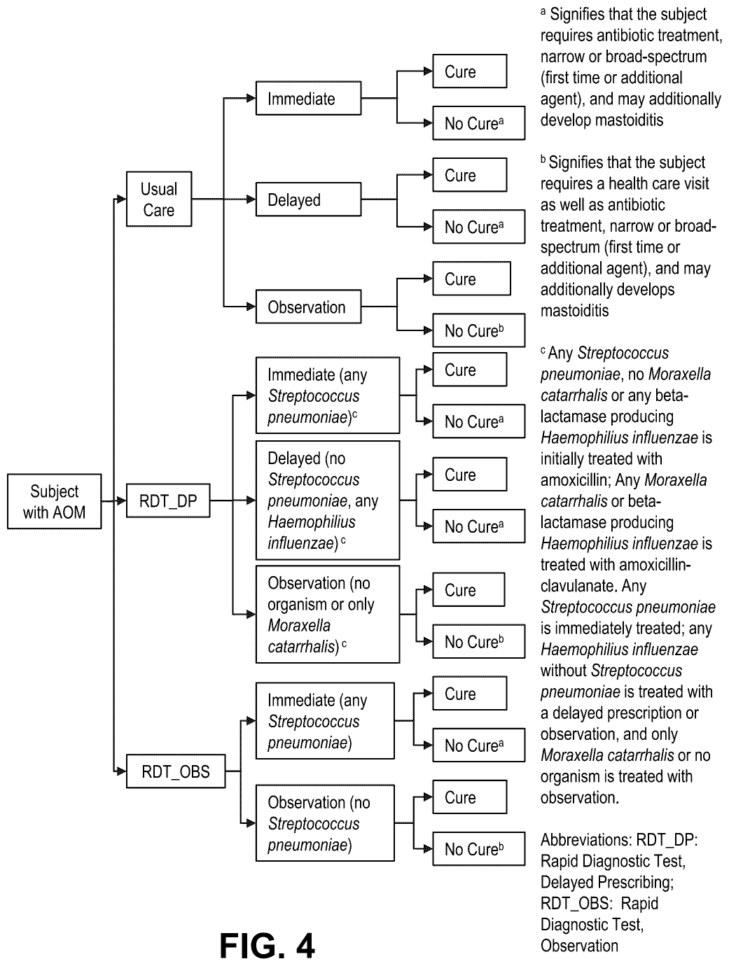

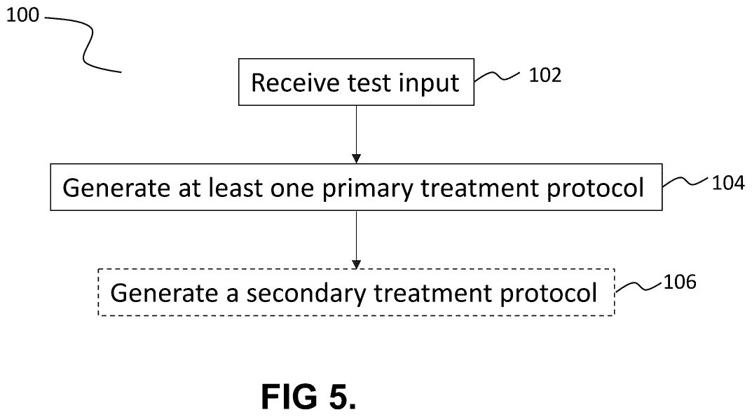

FIG. 4 depicts decision algorithms for treatment options in subjects with otitis media, or in the embodiment of FIG. 4, acute otitis media (AOM). In a first decision algorithm, treatment options under the usual standard of care are depicted. The usual standard of care intends the approach of care currently and commonly provided by medical providers, where a course of treatment is recommended irrespective of otopathogen(s) present in a subject presenting with clinical symptoms of otitis media or AOM. In the usual standard of care, otopathogen(s) causing the clinical symptoms are not typically known, as no diagnostic assay is performed to identify otopathogen(s) present in a biological sample taken from the subject. Thus, based on clinical symptoms and perhaps other factors apparent to the medical provider the treatment options include: (a) an antibiotic is prescribed with the intent to be filled and/or taken by the subject as soon as practicable (“immediate” treatment option), (b) an antibiotic is prescribed with the intent to be filled and taken if the patient worsens or does not improve within a designated time frame; this option (b) is sometimes referred to as a safety-net prescription or categorized as watchful waiting (“delayed” treatment option), or (c) no antibiotic is prescribed or indicated; instead a follow-up is conducted, the follow-up can be in-person or virtual (remote), and can be initiated by the clinician or clinician team or by the subject contacting the clinician or clinician team if the subject worsens or does not improve within a designated time frame (“observation” treatment option). For each of options (a). (b) and (c) an outcome of cure or no cure can occur. For subjects indicated for immediate treatment or delayed treatment, a no-cure outcome signifies that the subject requires antibiotic treatment, either a narrow-spectrum or a broad-spectrum antibiotic (first time or additional agent), and that the subject may additionally develop mastoiditis. For subjects indicated for observation, a no-cure outcome signifies that the subject requires a health care visit (in-person or remote) as well as antibiotic treatment, either a narrow or broad-spectrum antibiotic (first time or additional agent), and that the subject may additionally develop mastoiditis.

With continued reference to FIG. 4, a second algorithm is depicted for a subject with an otitis media or AOM diagnosis from a diagnostic test assay, such as a molecular assay or an antibody based assay, and treated with a delayed treatment option (the algorithm abbreviated in FIG. 4 as “RDT-DP”). Subjects given a delayed treatment protocol divide into categories based on otopathogen present (as described above in FIGS. 1-3): (d) if Streptococcus pneumoniae is present, then an immediate treatment is indicated; (e) if no Streptococcus pneumoniae is present, but Haemophilus influenzae (either subtype) is present, then delayed treatment is indicated; and (f) if no otopathogen is present or if only Moraxella catarrhalis is present, then observation treatment is indicated. For each of options (d), (e) and (f) an outcome of cure or no cure can occur. For subjects indicated for immediate treatment or delayed treatment (options (d) and (e)), a no-cure outcome signifies that the subject requires antibiotic treatment, either a narrow or broad-spectrum antibiotic (first time or additional agent), and that the subject may additionally develop mastoiditis. For subjects indicated for observation (option (f)), a no-cure outcome signifies that the subject requires a health care visit (in-person or remote) as well as antibiotic treatment, either a narrow or broad-spectrum antibiotic (first time or additional agent), and that the subject may additionally develop mastoiditis.

Continuing with FIG. 4, subjects with an otitis media or AOM diagnosis from a diagnostic test assay, such as a molecular assay or an antibody based assay, and treated with an “observation” treatment option (abbreviated in FIG. 4 as “RDT-OBS”), can be classified more finely based on otopathogen present: (g) if Streptococcus pneumoniae is present, then an immediate treatment is indicated; or (h) if no Streptococcus pneumoniae is present, then an observation treatment option is indicated. For subjects indicated for immediate treatment (option (g)), a no-cure outcome signifies that the subject requires antibiotic treatment, either a narrow or broad-spectrum antibiotic (first time or additional agent), and that the subject may additionally develop mastoiditis. For subjects indicated for observation (option (h)), a no-cure outcome signifies that the subject requires a health care visit (in-person or remote) as well as antibiotic treatment, either a narrow or broad-spectrum antibiotic (first time or additional agent), and that the subject may additionally develop mastoiditis.

Example 1 details a study using the algorithms of FIG. 4 and illustrating the individualized care achieved by the algorithms described herein.

In an embodiment, the subject with otitis media or with AOM is a child or

a pediatric patient, intending a person under the age of 21. In other embodiments, the subject is an infant, with an age between birth and 2 years, a child aged from 2 to 12 years, or an adolescent with an age of from 12 to 21 years. In other embodiments, the subject is an adolescent with an age from 11 to 21 years, which can be further classified into early (ages 11-14 years), middle (ages 15-17 years), and late (ages 18-21 years) adolescence.

In an embodiment, the time frame or “designated time frame” or “certain time frame” mentioned with respect to the methods and algorithms described herein is about 8 hours, 12 hours, 24 hours, 36 hours, 48 hours, 60 hours, 72 hours, or 96 hours. In other embodiments, the time frame or “designated time frame” or “certain time frame” mentioned with respect to the methods and algorithms described herein is not more than any of about 8 hours, 12 hours, 24 hours, 36 hours, 48 hours, 60 hours, or 72 hours. It will be appreciated that the time frame determined by a medical provider will depend on a number of factors, such as but not limited to, the age of the subject, relevant medical history, current health, length of time of current clinical symptoms, and/or severity of clinical symptoms.

It will be appreciated that the algorithms or portions thereof may be performed by a computing device. The computing device may be a mobile device, a point-of-care instrument, a hand-held instrument, or a diagnostic and/or treatment system.

FIG. 5 is a flow diagram of an example diagnostic and treatment method 100 performed by a computing device. At step 102, a diagnostic and treatment system receives test input. The test input may include the results of an assay described herein. For example, the test input may include information on the presence of absence of positive beta-lactamase H. influenzae, negative beta-lactamase H. influenzae, S. pneumoniae, and M. catarrhalis in a patient sample. The test input may be automatically transferred to the diagnostic and treatment system, such as directly from a device performing an assay, or a software program running on the assay device, such as via file transfer. Additionally or alternatively, the test input may be manually entered into the diagnostic and treatment system, such as via typing on a keyboard.

In step 104, the diagnostic and treatment system presents at least one primary treatment option or protocol. The at least one primary treatment option is generated by a treatment model based on the test input. The treatment model may be implemented using various algorithms. For example, the treatment model may apply a decision algorithm as depicted in any one FIGS. 1-4 or Tables 1-1 to 3-2, or any portions or any combinations thereof. In some implementations, when more than one treatment option is presented, the treatment model may present treatment options in ranked order, which may be based on likelihood of treatment success. In some implementations, when more than one treatment option is presented, the treatment model may present alternatives based on additional factors, such as whether one proposed treatment option may elicit an allergic response in some patients or a recommended antibiotic may have limited availability or higher expense. The at least one treatment option may be presented on a user interface and is directly or indirectly provided to a clinician, medical caregiver, patient, and/or pharmacist.

At optional step 106, the diagnostic and treatment system presents a secondary treatment option. Additional operations may immediately precede step 106, such as the diagnostic and treatment system receiving follow-on input. The follow-on input may include, for example, information about whether a patient complied with the primary treatment option, whether a patient's symptoms have improved or have worsened following implementation of the primary treatment option, and the amount of time elapsed since provision of or patient compliance with the primary treatment option. In one example, the secondary treatment option may be a rescue antibiotic as described herein.

Each of the step 104 and optional step 106 may be presented on an output interface, such as a digital output interface, for example a website, mobile device, or point-of-care instrument.

The tables below (i.e., Tables 1-1, 1-2, 2-1, 2-2, 3-1, and 3-2) provide additional details on the diagnostic test algorithms. All algorithms may be used in conjunction with clinical decision-making based on a patient's clinical presentation and relevant medical history including but not limited to the following: immunocompromised status, history of recent acute otitis media episodes or recurrent acute otitis media, recent antibiotic use, history of tympanostomy tube placement, otorrhea, concurrent conjunctivitis, severity of infection, medication allergy history, and access to follow-up care if needed.

Observation or delayed prescribing may be considered alternative options to immediate antibiotic therapy regardless of organisms detected given the high spontaneous resolution rates and low complication rates of untreated acute otitis media. In the case of use of observation or delayed prescribing, the rapid diagnostic testing algorithms can be used to guide antibiotic agent choice.

TABLE 1-1
Rapid Diagnostic Test Algorithm 1

| | Optimal | | Preferred Rescue² |
|---|---|---|---|
| Bacterial Otopathogens¹ | Management | Preferred Initial | Antibiotic for |
| Detected | Strategy | Antibiotic Type | Treatment Failure |
| None | Observation³ | None | Narrow-spectrum may |
| | | | not require beta- |
| | | | lactamase activity⁴ |
| S. pneumoniae | Immediate⁵ | Narrow-spectrum does | Broader-spectrum |
| | | not require beta- | with beta-lactamase |
| | | lactamase activity | activity⁶ |
| S. pneumoniae + non-beta | Immediate | Narrow-spectrum does | Broader-spectrum |
| lactamase producing | | not require beta- | with beta-lactamase |
| H. influenzae | | lactamase activity | activity |
| S. pneumoniae + non-beta | Immediate | Broader-spectrum with | Broader-spectrum |
| lactamase producing | | beta-lactamase activity | with beta-lactamase |
| H. influenzae + M. catarrhalis | | | activity |
| S. pneumoniae + beta | Immediate | Broader-spectrum with | Broader-spectrum |
| lactamase producing | | beta-lactamase activity | with beta-lactamase |
| H. influenzae | | | activity |
| S. pneumoniae + beta | Immediate | Broader-spectrum with | Broader-spectrum |
| lactamase producing | | beta-lactamase activity | with beta-lactamase |
| H. influenzae + M. catarrhalis | | | activity |
| S. pneumoniae + | Immediate | Broader-spectrum with | Broader-spectrum |
| M. catarrhalis | | beta-lactamase activity | with beta-lactamase |
| | | | activity |
| H. influenzae non-beta | Delayed⁷ | Narrow-spectrum does | Broader-spectrum |
| lactamase producing | | not require beta- | with beta-lactamase |
| | | lactamase activity | activity |
| H. influenzae non-beta | Delayed | Broader-spectrum with | Broader-spectrum |
| lactamase producing + | | beta-lactamase activity | with beta-lactamase |
| M. catarrhalis | | | activity |
| H. influenzae beta | Delayed | Broader-spectrum with | Broader-spectrum |
| lactamase producing | | beta-lactamase activity | with beta-lactamase |
| | | | activity |
| H. influenzae beta | Delayed | Broader-spectrum with | Broader-spectrum |
| lactamase producing + | | beta-lactamase activity | with beta-lactamase |
| M. catarrhalis | | | activity |
| M. catarrhalis | Observation | None | Broader-spectrum |
| | | | with beta-lactamase |
| | | | activity |

¹Includes S. pneumoniae, H. influenzae, M. catarrhalis.
²An antibiotic to be given if a patient worsens or does not improve within a designated time frame from initial treatment.
³No antibiotic indicated, follow-up in-person or by contacting the clinician or clinician team if patient worsens or does not improve within a designated time frame. Could consider a delayed antibiotic prescription with rescue antibiotic preference as an alternative option. Sometimes referred to as watchful waiting.
⁴Refer to antibiotic Table A.
⁵An antibiotic prescribed with the intent to be filled and/or taken by the patient right away.
⁶Refer to antibiotic Table A.
⁷An antibiotic prescribed with the intent to be filled and taken if the patient worsens or does not improve within a designated time frame. Sometimes referred to as a safety-net prescription or categorized as watchful waiting.

TABLE 1-2
Rapid Diagnostic Test Algorithm 1

| | | | Does rescue² |
|---|---|---|---|
| | | Does initial | antibiotic for |
| | | antibiotic agent | treatment |
| | | require activity | failure require |
| | | against | activity against |
| | Optimal | organism(s) that | organism(s) that |
| | Management | produce beta- | produce beta- |
| Bacterial Otopathogens¹ Detected | Strategy | lactamase? | lactamase? |
| None | Observation³ | N/A | No⁴ |
| S. pneumoniae | Immediate⁵ | No | Yes⁶ |
| S. pneumoniae + non-beta lactamase | Immediate | No | Yes |
| producing H. influenzae | | | |
| S. pneumoniae + non-beta lactamase | Immediate | Yes | Yes |
| producing H. influenzae + | | | |
| M. catarrhalis | | | |
| S. pneumoniae + beta lactamase | Immediate | Yes | Yes |
| producing H. influenzae | | | |
| S. pneumoniae + beta lactamase | Immediate | Yes | Yes |
| producing H. influenzae + | | | |
| M. catarrhalis | | | |
| S. pneumoniae + M. catarrhalis | Immediate | Yes | Yes |
| H. influenzae non-beta lactamase | Delayed⁷ | No | Yes |
| producing | | | |
| H. influenzae non-beta lactamase | Delayed | Yes | Yes |
| producing + M. catarrhalis | | | |
| H. influenzae beta lactamase producing | Delayed | Yes | Yes |
| H. influenzae beta lactamase producing + | Delayed | Yes | Yes |
| M. catarrhalis | | | |
| M. catarrhalis | Observation | N/A | Yes |

¹Includes S. pneumoniae, H. influenzae, M. catarrhalis.
²An antibiotic to be given if a patient worsens or does not improve within a designated time frame from initial treatment.
³No antibiotic indicated, follow-up in-person or by contacting the clinician or clinician team if patient worsens or does not improve within a designated time frame. Could consider a delayed antibiotic prescription with rescue antibiotic preference as an alternative option. Sometimes referred to as watchful waiting.
⁴Refer to antibiotic Table A.
⁵An antibiotic prescribed with the intent to be filled and/or taken by the patient right away.
⁶Refer to antibiotic Table A.
⁷An antibiotic prescribed with the intent to be filled and taken if the patient worsens or does not improve within a designated time frame. Sometimes referred to as a safety-net prescription or categorized as watchful waiting.

TABLE 2-1
Rapid Diagnostic Test Algorithm 2

| | Optimal | | Preferred Rescue |
|---|---|---|---|
| | Management | Preferred Initial | Antibiotic² for |
| Bacterial Otopathogens¹ Detected | Strategy | Antibiotic Type | Treatment Failure |
| None | Observation³ | None | Narrow-spectrum |
| | | | may not require |
| | | | beta-lactamase |
| | | | activity⁴ |
| S. pneumoniae | Immediate⁵ | Narrow-spectrum | Broader-spectrum |
| | | does not require | with beta- |
| | | beta-lactamase | lactamase activity⁶ |
| | | activity | |
| S. pneumoniae + non-beta | Immediate | Narrow-spectrum | Broader-spectrum |
| lactamase producing H. influenzae | | does not require | with beta- |
| | | beta-lactamase | lactamase activity |
| | | activity | |
| S. pneumoniae + non-beta | Immediate | Broader-spectrum | Broader-spectrum |
| lactamase producing H. influenzae + | | with beta- | with beta- |
| M. catarrhalis | | lactamase activity | lactamase activity |
| S. pneumoniae + beta lactamase | Immediate | Broader-spectrum | Broader-spectrum |
| producing H. influenzae | | with beta- | with beta- |
| | | lactamase activity | lactamase activity |
| S. pneumoniae + beta lactamase | Immediate | Broader-spectrum | Broader-spectrum |
| producing H. influenzae + | | with beta- | with beta- |
| M. catarrhalis | | lactamase activity | lactamase activity |
| S. pneumoniae + M. catarrhalis | Immediate | Broader-spectrum | Broader-spectrum |
| | | with beta- | with beta- |
| | | lactamase activity | lactamase activity |
| H. influenzae non-beta lactamase | Observation | None | Narrow-spectrum |
| producing | | | does not require |
| | | | beta-lactamase |
| | | | activity |
| H. influenzae non-beta lactamase | Observation | None | Broader-spectrum |
| producing + M. catarrhalis | | | with beta- |
| | | | lactamase activity |
| H. influenzae beta lactamase | Observation | None | Broader-spectrum |
| producing | | | with beta- |
| | | | lactamase activity |
| H. influenzae beta lactamase | Observation | None | Broader-spectrum |
| producing + M. catarrhalis | | | with beta- |
| | | | lactamase activity |
| M. catarrhalis | Observation | None | Broader-spectrum |
| | | | with beta- |
| | | | lactamase activity |

¹Includes S. pneumoniae, H. influenzae, M. catarrhalis.
²An antibiotic to be given if a patient worsens or does not improve within a designated time frame from initial treatment.
³No antibiotic indicated, follow-up in-person or by contacting the clinician or clinician team if patient worsens or does not improve within a designated time frame. Could consider a delayed antibiotic prescription with rescue antibiotic preference as an alternative option. Sometimes referred to as watchful waiting.
⁴Refer to antibiotic Table A.
⁵An antibiotic prescribed with the intent to be filled and/or taken by the patient right away.
⁶Refer to antibiotic Table A.

TABLE 2-2
Rapid Diagnostic Test Algorithm 2

| | | Does initial | Does rescue² |
|---|---|---|---|
| | | antibiotic agent | antibiotic for |
| | | require activity | treatment failure |
| | | against | require activity |
| | | organism(s) | against |
| | Optimal | that produce | organism(s) that |
| | Management | beta- | produce beta- |
| Bacterial Otopathogens¹ Detected | Strategy | lactamase? | lactamase? |
| None | Observation³ | N/A | No⁴ |
| S. pneumoniae | Immediate⁵ | No | Yes⁶ |
| S. pneumoniae + non-beta lactamase | Immediate | No | Yes |
| producing H. influenzae | | | |
| S. pneumoniae + non-beta lactamase | Immediate | Yes | Yes |
| producing H. influenzae + | | | |
| M. catarrhalis | | | |
| S. pneumoniae + beta lactamase | Immediate | Yes | Yes |
| producing H. influenzae | | | |
| S. pneumoniae + beta lactamase | Immediate | Yes | Yes |
| producing H. influenzae + | | | |
| M. catarrhalis | | | |
| S. pneumoniae + M. catarrhalis | Immediate | Yes | Yes |
| H. influenzae non-beta lactamase | Observation | N/A | No |
| producing | | | |
| H. influenzae non-beta lactamase | Observation | N/A | Yes |
| producing + M. catarrhalis | | | |
| H. influenzae beta lactamase | Observation | N/A | Yes |
| producing | | | |
| H. influenzae beta lactamase | Observation | N/A | Yes |
| producing + M. catarrhalis | | | |
| M. catarrhalis | Observation | N/A | Yes |

¹Includes S. pneumoniae, H. influenzae, M. catarrhalis.
²An antibiotic to be given if a patient worsens or does not improve within a designated time frame from initial treatment.
³No antibiotic indicated, follow-up in-person or by contacting the clinician or clinician team if patient worsens or does not improve within a designated time frame. Could consider a delayed antibiotic prescription with rescue antibiotic preference as an alternative option. Sometimes referred to as watchful waiting.
⁴Refer to antibiotic Table A.
⁵An antibiotic prescribed with the intent to be filled and/or taken by the patient right away.
⁶Refer to antibiotic Table A.

TABLE 3-1
Rapid Diagnostic Testing Algorithm 3

| | Optimal | | |
|---|---|---|---|
| Bacterial Otopathogens¹ | Management | Preferred Initial Antibiotic | Preferred Rescue Antibiotic² |
| Detected | Strategy | Type | for Treatment Failure |
| None | Observation³ | None | Narrow-spectrum may not |
| | | | require beta-lactamase |
| | | | activity⁴ |
| S. pneumoniae | Immediate⁵ | Narrow-spectrum does not | Broader-spectrum with beta- |
| | | require beta-lactamase | lactamase activity⁶ |
| | | activity | |
| S. pneumoniae + non- | Immediate | Narrow-spectrum does not | Broader-spectrum with beta- |
| beta lactamase | | require beta-lactamase | lactamase activity |
| producing H. influenzae | | activity | |
| S. pneumoniae + non- | Immediate | Broader-spectrum with | Broader-spectrum with beta- |
| beta lactamase | | beta-lactamase activity | lactamase activity |
| producing H. influenzae + | | | |
| M. catarrhalis | | | |
| S. pneumoniae + beta | Immediate | Broader-spectrum with | Broader-spectrum with beta- |
| lactamase producing | | beta-lactamase activity | lactamase activity |
| H. influenzae | | | |
| S. pneumoniae + beta | Immediate | Broader-spectrum with | Broader-spectrum with beta- |
| lactamase producing | | beta-lactamase activity | lactamase activity |
| H. influenzae + | | | |
| M. catarrhalis | | | |
| S. pneumoniae + | Immediate | Broader-spectrum with | Broader-spectrum with beta- |
| M. catarrhalis | | beta-lactamase activity | lactamase activity |
| H. influenzae non-beta | Delayed⁷ | Narrow-spectrum does not | Broader-spectrum with beta- |
| lactamase producing | | require beta-lactamase | lactamase activity |
| | | activity | |
| H. influenzae non-beta | Delayed | Broader-spectrum with | Broader-spectrum with beta- |
| lactamase producing + | | beta-lactamase activity | lactamase activity |
| M. catarrhalis | | | |
| H. influenzae beta | Delayed | Broader-spectrum with | Broader-spectrum with beta- |
| lactamase producing | | beta-lactamase activity | lactamase activity |
| H. influenzae beta | Delayed | Broader-spectrum with | Broader-spectrum with beta- |
| lactamase producing + | | beta-lactamase activity | lactamase activity |
| M. catarrhalis | | | |
| M. catarrhalis | Delayed | Broader-spectrum with | Broader-spectrum with beta- |
| | | beta-lactamase activity | lactamase activity |

¹Includes S. pneumoniae, H. influenzae, M. catarrhalis.
²An antibiotic to be given if a patient worsens or does not improve within a designated time frame from initial treatment.
³No antibiotic indicated, follow-up in-person or by contacting the clinician or clinician team if patient worsens or does not improve within a designated time frame. Could consider a delayed antibiotic prescription with rescue antibiotic preference as an alternative option. Sometimes referred to as watchful waiting.
⁴Refer to antibiotic Table A.
⁵An antibiotic prescribed with the intent to be filled and/or taken by the patient right away.
⁶Refer to antibiotic Table A.
⁷An antibiotic prescribed with the intent to be filled and taken if the patient worsens or does not improve within a designated time frame; sometimes referred to as a safety-net prescription or categorized as watchful waiting.

TABLE 3-2
Rapid Diagnostic Testing Algorithm 3

| | | | Does rescue² |
|---|---|---|---|
| | | Does initial | antibiotic for |
| | | antibiotic agent | treatment failure |
| | | require activity | require activity |
| | | against | against |
| | Optimal | organism(s) that | organism(s) that |
| Bacterial Otopathogens¹ | Management | produce beta- | produce beta- |
| Detected | Strategy | lactamase? | lactamase? |
| None | Observation³ | N/A | No⁴ |
| S. pneumoniae | Immediate⁵ | No | Yes⁶ |
| S. pneumoniae + non-beta | Immediate | No | Yes |
| lactamase producing | | | |
| H. influenzae | | | |
| S. pneumoniae + non-beta | Immediate | Yes | Yes |
| lactamase producing | | | |
| H. influenzae + M. catarrhalis | | | |
| S. pneumoniae + beta lactamase | Immediate | Yes | Yes |
| producing H. influenzae | | | |
| S. pneumoniae + beta lactamase | Immediate | Yes | Yes |
| producing H. influenzae + | | | |
| M. catarrhalis | | | |
| S. pneumoniae + M. catarrhalis | Immediate | Yes | Yes |
| H. influenzae non-beta lactamase | Delayed⁷ | No | Yes |
| producing | | | |
| H. influenzae non-beta lactamase | Delayed | Yes | Yes |
| producing + M. catarrhalis | | | |
| H. influenzae beta lactamase | Delayed | Yes | Yes |
| producing | | | |
| H. influenzae beta lactamase | Delayed | Yes | Yes |
| producing + M. catarrhalis | | | |
| M. catarrhalis | Delayed | Yes | Yes |

¹Includes S. pneumoniae, H. influenzae, M. catarrhalis
²An antibiotic to be given if a patient worsens or does not improve within a designated time frame from initial treatment
³No antibiotic indicated, follow-up in-person or by contacting the clinician or clinician team if patient worsens or does not improve within a designated time frame. Could consider a delayed antibiotic prescription with rescue antibiotic preference as an alternative option. Sometimes referred to as watchful waiting.
⁴Refer to antibiotic Table A.
⁵An antibiotic prescribed with the intent to be filled and/or taken by the patient right away.
⁶Refer to antibiotic Table A.
⁷An antibiotic prescribed with the intent to be filled and taken if the patient worsens or does not improve within a designated time frame. Sometimes referred to as a safety-net prescription or categorized as watchful waiting.

The algorithms disclosed herein may help a medical care provider determine a treatment protocol for a patient presenting with symptoms of otitis media. The present standard of care does not include identifying the otopathogen(s) causing clinical symptoms of otitis media, nor does it include determining a treatment protocol based on the otopathogen(s) causing clinical symptoms of otitis media. Compared to the current standard of care, the present disclosure enables clinicians to make treatment decisions informed by the presence of absence of various otopathogen(s). The present disclosure enables clinicians to not issue a prescription or to issue a prescription for immediate or delayed antibiotic treatment, as described herein, based on test results as incorporated into the provided algorithms.

### IV. Methods of Treatment

The samples, assays, and/or algorithms disclosed herein may form the basis of treating a patient having otitis media. Treatment includes one or more of an immediate antibiotic, a delayed antibiotic, a rescue antibiotic, no antibiotic, observation, and follow-up, each as disclosed herein. An antibiotic may be with or without beta-lactamase activity.

In one example, a nasal sample obtained from a patient is combined with an extraction reagent to produce a test sample. The test sample, or a portion thereof, may be combined with one or more reagents, such as an amplification reagent, and such as to produce an amplification sample. The amplification sample may be subjected to an assay disclosed herein, such as an amplification-based assay that amplifies otopathogenic nucleic acid(s), if present. The amplified nucleic acid, if present, may then be detected. The detection result may be subjected to one or more of the algorithms disclosed herein to guide a treatment protocol. The subject may then be treated for otitis media.

### V. Kits

Kits disclosed herein include components for use in one or more of the assays disclosed herein. For example, a kit may be a kit suitable for a PCR-based assay and may include primers or probes complementary to a DNA target (such as otopathogenic nucleic acids). Primers may be provided as a set including at least one forward primer and at least one reverse primer complementary to a DNA target. The kit may also include one or more of a DNA polymerase and deoxynucleoside triphosphates (dNTPs) present in a suitable buffer for nucleic acid amplification and detection. The primers or probes may be specific to one or more of H. influenzae, M. catarrhalis, or S. pneumonia. The primers or probes may be specific to one or more of beta-lactamase producing H. influenzae and non-beta-lactamase producing H. influenza. The primers or probes may be specific for one or more antimicrobial resistance markers or genes, such as ctx-M (blaCTX-M, subgroup 1), kpc (blaKPC), mecA, ndm (blaNDM), oxa-23 (blaOXA-23), oxa-24 (blaOXA-24), oxa-48 (blaOXA-48), oxa-58 (blaOXA-58), tem (blaTEM), or vim (blaVIM). In some embodiments, a kit includes primers specific to all of beta-lactamase producing H. influenzae, non-beta-lactamase producing H. influenzae, M. catarrhalis, and S. pneumonia. More than one set of primers or probes capable of amplifying one organism may be included in a kit.

The present standard of care does not include identifying the otopathogen(s) causing clinical symptoms of otitis media. Instead, treatment decisions are made in the absence of information about which otopathogen(s) are present and which are absent in a patient. Compared to the standard of care, the presently disclosed kits, and related assays, enable the easy and rapid identification of likely otopathogen(s) responsible for otitis media. The presently disclosed kits, and related assays, combine primers or probes directed to identifying common otopathogen(s) responsible for otitis media, or the otopathogen(s) whose presence or absence has a notable impact on a treatment decision or protocol. Such otopathogen(s) may include beta-lactamase producing H. influenzae, non-beta-lactamase producing H. influenzae, M. catarrhalis, and S. pneumonia.

### IV. Examples

The following examples are illustrative in nature and are in no way intended to be limiting.

### Example—Nasopharyngeal Testing to Individualize Management of Acute Otitis Media (AOM)

This example demonstrates that testing of nasopharyngeal (NP) samples using a polymerase chain reaction based assay (an example of a rapid diagnostic test or RDT) can effectively exclude the presence of organisms in middle ear fluid and is useful to individualize care.

Two algorithms for AOM management based on NP bacterial otopathogens were developed, and are depicted in FIG. 4. The algorithms provide recommendations on prescribing strategy (immediate, delayed, or observation) and antimicrobial agent. A tiering system based on expected pathogen-associated severity and resistance was used. The primary outcome was the incremental cost-effectiveness ratio expressed as cost per quality adjusted life day gained. A decision-analytic model was used to evaluate the cost-effectiveness of the rapid diagnostic test algorithms compared to usual care from a societal perspective over a 30-day time horizon. Secondary outcomes included the (1) cost at which a rapid diagnostic test would be cost-effective and (2) potential reduction in annual antibiotics used.

The algorithm that used immediate, delayed prescribing, and observation based on pathogen (FIG. 4, algorithm “RDT-DP”) had an incremental cost-effectiveness ratio of $1336.15/quality adjusted life day gained compared with usual care and strongly dominated the rapid diagnostic test that used only immediate prescribing and observation (FIG. 4, algorithm “RDT-OBS”). At a rapid diagnostic test cost of $278.56, the incremental cost-effectiveness ratio for the RDT-DP algorithm exceeded the willingness to pay threshold ($274 per quality adjusted life day gained); however, if the cost of the rapid diagnostic test was <$212.10, the incremental cost-effectiveness ratio was below the threshold. Both algorithms (RDT-DP and RDT-OBS) reduced annual antibiotic use, including broad-spectrum use, compared to usual care—e.g., RDT-DP 4.7 million doses (56% reduction) and RDT-OBS 5.4 million doses (49% reduction), compared to usual care of 10.5 million doses. The use of a rapid diagnostic test for AOM is cost-effective and substantially reduces unnecessary antibiotic use.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

It is claimed:

1. A kit comprising:
a plurality of primers specific to each of beta-lactamase producing H. influenzae, non-beta-lactamase producing H. influenzae, M. catarrhalis, and S. pneumonia,
wherein the primers specific to beta-lactamase producing H. influenzae and non-beta-lactamase producing H. influenzae are capable of amplifying at least one gene that encodes beta-lactamase.

2. The kit of claim 1, wherein the at least one gene that encodes beta-lactamase is selected from ctx-M (blaCTX-M, subgroup 1), kpc (blaKPC), mecA, ndm (blaNDM), oxa-23 (blaOXA-23), oxa-24 (blaOXA-24), oxa-48 (blaOXA-48), oxa-58 (blaOXA-58), tem (blaTEM), and vim (blaVIM).

3. The kit of claim 1 or claim 2, further comprising at least one DNA polymerase and a plurality of deoxynucleoside triphosphates (dNTPs).

4. A method to determine a treatment protocol in a subject clinically diagnosed with otitis media, comprising:
analyzing a test result indicating presence or absence of negative beta-lactamase H. influenzae, positive beta-lactamase H. influenzae, M. catarrhalis, and S. pneumoniae from a biological sample from the subject to determine if S. pneumonia is present or absent, wherein
(a) if S. pneumonia is present, determining (i) if one or both of M. catarrhalis and positive beta-lactamase H. influenzae are present, wherein if one or both are present, then recommending treatment with an immediate antibiotic with beta-lactamase activity, (ii) if negative beta-lactamase producing H. influenzae is present, then recommending treatment with an immediate antibiotic that does not require beta lactamase activity, and/or (iii) if none of positive beta-lactamase H. influenzae, negative beta-lactamase H. influenzae, and M. catarrhalis are present then recommending treatment with an immediate antibiotic that does not require beta lactamase activity; and
(b) if S. pneumonia is not present, recommending no immediate antibiotic treatment.

5. The method of claim 4, wherein (b) further comprises determining if negative beta-lactamase H. influenzae, positive beta-lactamase H. influenzae and M. catarrhalis are present, wherein if negative beta-lactamase H. influenzae, positive beta-lactamase H. influenzae are absent and M. catarrhalis is present, recommending (i) follow-up if clinical symptoms do not improve or (ii) a delayed prescription for an antibiotic with beta-lactamase activity.

6. The method of claim 5, wherein (i) further comprises wherein in the event clinical symptoms do not improve, a rescue antibiotic is provided, wherein the rescue antibiotic has beta lactamase activity.

7. The method of claim 4 or claim 5, wherein (b) further comprises determining if either positive beta-lactamase or negative beta-lactamase H. influenzae are present, wherein if positive beta-lactamase H. influenzae is present also determining if M. catarrhalis is present, wherein if positive beta-lactamase H. influenzae and/or M. catarrhalis are present, recommending (i) observation, with follow-up in clinical symptoms do not improve or (ii) a delayed prescription for an antibiotic with beta-lactamase activity.

8. The method of claim 7, wherein (i) further comprises providing rescue antibiotic in the event clinical symptoms do not improve, wherein the rescue antibiotic has beta lactamase activity.

9. The method of claim 7, wherein if positive beta-lactamase H. influenzae or M. catarrhalis are absent, recommending (i) follow-up if clinical symptoms do not improve or (ii) observation with a delayed prescription for an antibiotic that does not require beta-lactamase activity.

10. The method of claim 9, wherein (i) further comprises providing rescue antibiotic in the event clinical symptoms do not improve, wherein the rescue antibiotic does not require beta lactamase activity.

11. The method of claim 4, wherein (b) further comprises determining whether negative beta-lactamase H. influenzae, positive beta-lactamase H. influenzae and M. catarrhalis are present or absent, where if all are absent, recommending no immediate antibiotic treatment, observation, or a delayed prescription for an antibiotic that does not require beta-lactamase activity.

12. The method of claim 4, wherein (b) further comprises determining presence or absence of negative beta-lactamase H. influenzae, positive beta-lactamase H. influenzae, and M. catarrhalis wherein if (i) negative beta-lactamase H. influenzae, positive beta-lactamase H. influenzae, and M. catarrhalis are not present, recommending no immediate antibiotic treatment, or (ii) one or more of negative beta-lactamase H. influenzae, positive beta-lactamase H. influenzae, and M. catarrhalis are present then recommending observation, follow-up, and/or a delayed prescription for an antibiotic with beta-lactamase activity.

13. A method to reduce antibiotic usage in subjects pathogenically diagnosed with otitis media, where the pathogenic diagnosis comprises a determination of presence or absence of negative beta-lactamase H. influenzae, positive beta-lactamase H. influenzae, M. catarrhalis, and S. pneumoniae, comprising:
evaluating the pathogenic diagnosis to determine
(i) if S. pneumonia, negative beta-lactamase H. influenzae, and positive beta-lactamase H. influenzae are absent and M. catarrhalis is present, wherein if (i) is true, providing no immediate antibiotic treatment;
(ii) if S. pneumonia is absent and positive beta-lactamase H. influenzae or M. catarrhalis are present, wherein if (ii) is true, providing no immediate antibiotic treatment;
(iii) if S. pneumonia is present and M. catarrhalis and/or positive beta-lactamase H. influenzae is/are present, wherein if (iii) is true, providing immediate antibiotic treatment with an antibiotic with beta-lactamase activity;
(iv) if S. pneumonia and negative beta-lactamase H. influenzae are present, and M. catarrhalis and positive beta-lactamase H. influenzae are absent, wherein if (iv) is true, providing immediate antibiotic treatment with an antibiotic that does not require beta-lactamase activity.

14. The method of claim 13, further comprising evaluating to determine (v) if S. pneumonia is absent and neither of negative beta-lactamase H. influenzae nor M. catarrhalis are present, wherein if (v) is true, providing no immediate antibiotic treatment, and optionally recommending follow-up if not improvement in a defined time period.

15. The method of claim 14, wherein option (v) further comprises providing a rescue antibiotic that does not require beta lactamase activity.

16. The method of claim 14 or claim 15, wherein the time period is 24 hours, 36 hours, 48 hours, 72 hours or 96 hours.

17. The method of claim 13, wherein option (ii) further comprises providing or recommending to provide a delayed prescription for an antibiotic with beta-lactamase activity.

18. The method of any one of claims 13-17, wherein option (i) further comprises providing a rescue antibiotic with beta lactamase activity.

19. The method of any one of claims 14-18 wherein option (i) and/or option (ii) further comprise one or more of recommending observation, follow-up if not improving, a delayed prescription for an antibiotic with beta-lactamase activity.

20. A method for diagnosing and/or treating otitis media, comprising:
obtaining a test result from an assay performed on a biological sample from a subject that reports presence or absence of negative beta-lactamase H. influenzae, positive beta-lactamase H. influenzae, M. catarrhalis, and S. pneumoniae;
applying a decision algorithm to the test result to determine a treatment protocol;
administering, recommending administration of, or having administered the treatment protocol; and
monitoring patient response to the treatment protocol,
wherein the decision algorithm comprises evaluating the test result to determine one or more of the following:
(i) if S. pneumonia, negative beta-lactamase H. influenzae, and positive beta-lactamase are absent and M. catarrhalis is present;
(ii) if S. pneumonia is absent and positive beta-lactamase H. influenzae and/or M. catarrhalis are present;
(ii′) if S. pneumonia negative beta-lactamase H. influenzae, positive beta-lactamase H. influenzae, and M. catarrhalis are absent,
(iii) if S. pneumonia is present and M. catarrhalis and/or positive beta-lactamase H. influenzae is/are present;
(iv) if S. pneumonia and negative beta-lactamase H. influenzae are present, and M. catarrhalis and positive beta-lactamase H. influenzae are absent; and
(v) if S. pneumonia, negative beta-lactamase H. influenzae, positive beta-lactamase H. influenzae, and M. catarrhalis are absent.

21. The method of claim 20, wherein
if (i), (ii) or (ii′) are true, providing no immediate antibiotic treatment;
if (iii) is true, providing immediate antibiotic treatment with an antibiotic with beta-lactamase activity; and/or
if (iv) is true, providing immediate antibiotic treatment with an antibiotic that does not require beta-lactamase activity.

22. The method of claim 21, wherein if (i) or (ii) are true, recommending a treatment option selected from the group consisting of (x) observation with clinical follow-up and (y) providing a delayed prescription with appropriate clinical follow-up.

23. The method of claim 22, wherein after said observation with clinical follow-up if the patient worsens or does not improve a rescue antibiotic treatment is provided.

24. The method of claim 23, wherein the rescue antibiotic has beta lactamase activity, if positive beta-lactamase H. influenzae is present and/or M. catarrhalis is present.

25. The method of any one of claims 20-24, wherein the steps of obtaining and applying are performed by a device, including a mobile device, a point of care instrument, a hand-held instrument, a diagnostic system, or a computer.

26. The method of any one of claims 20-24, wherein the steps of obtaining and applying are performed by a human, optionally where in the algorithm is electronically presented on a screen or is a hand-held written instrument.

27. A computer-implemented method for diagnosing otitis media, comprising:
receiving a test result from an assay performed on a biological sample from a subject, where in the test result reports presence or absence of positive beta-lactamase H. influenzae, negative beta-lactamase H. influenzae, S. pneumoniae, and M. catarrhalis;
applying a decision algorithm to the test result to determine a treatment protocol,
providing the treatment protocol to the subject, a medical caregiver, clinician, or pharmacist, wherein the decision algorithm comprises:
(i) determining if S. pneumonia is present or absent,
wherein if present, then:
(a) determining if M. catarrhalis and/or positive beta-lactamase H. influenzae is/are present,
(b) if (a) is false, determine if negative beta-lactamase H. influenzae is present or absent;
wherein if absent, then optionally determining
(c) if negative beta-lactamase H. influenzae and positive beta-lactamase H. influenzae are absent and M. catarrhalis is present;
(d) if positive beta-lactamase H. influenzae and/or M. catarrhalis is/are present; or
(e) if negative beta-lactamase H. influenzae and M. catarrhalis are absent;
wherein if (a) is true, providing immediate antibiotic treatment with an antibiotic with beta-lactamase activity;
wherein if (b) is true, providing immediate antibiotic treatment with an antibiotic that does not require beta-lactamase activity
optionally, wherein if (c), (d) or (e) is true, providing no immediate antibiotic treatment.

28. A diagnostic system for performing the method of claim 27, comprising:
a storage component for storing data, wherein the storage component optionally has instructions for receiving a test result;
a processor for processing data, wherein the processor is coupled to the storage component and configured to execute the instructions to receive the test rest and analyze it according to an algorithm, and
a display component for displaying information regarding the analysis and/or a print or share functionality to convey information regarding the analysis.

29. The method of claim 27 or claim 28, wherein a test result is placed on the storage component via a human via keyboard entry or via file transfer.

30. The method of any one of claims 27-29, wherein the processor is a computer process, or wherein the processor is on a mobile device.

31. The method of any one of claims 27-30, wherein said determining comprises determining for a fluid sample from the subject.

32. The method of claim 31, wherein the fluid sample is sputum, oropharyngeal, saliva, middle or inner ear fluid, or a nasal sample.

33. The method of claim 32, wherein the nasal sample is a nasopharyngeal sample, an anterior naris sample, an inferior turbinate sample, a middle turbinate sample, or a superior turbinate sample.

34. The method of any one of claims 27-33, wherein the computer-implemented method is performed on a mobile device, a computer, a server, or a diagnostic instrument.

35. The method of any one of claims 27-34, wherein the positive beta-lactamase H. influenzae is selected from the H. influenzae isolates TEM-1 and ROB-1.

36. The method of any one of claims 27-35, further comprising analyzing, evaluating or determining presence or absence of an antimicrobial resistance marker.

37. The method of claim 36, wherein the antimicrobial resistance marker is selected from ctx-M (blaCTX-M, subgroup 1), kpc (blaKPC), mecA, ndm (blaNDM), oxa-23 (blaOXA-23), oxa-24 (blaOXA-24), oxa-48 (blaOXA-48), oxa-58 (blaOXA-58), tem (blaTEM), vim (blaVIM).

38. The method of claim 36 or claim 37, wherein said analyzing, evaluating or determining comprises determining presence or absence of a viral pathogen.

39. The method of claim 38, wherein the vial pathogen is selected from Influenza A, Influenza B, Rhinovirus, SARS-CoV-2, a rhinovirus, middle eastern respiratory syndrome-coronavirus, a coronavirus, a respiratory syncytial virus (RSV).

40. The method of claim 38 or claim 39, wherein a relative qualitative determination of each detected pathogen is made or wherein a quantitative determination of one or more pathogens is made.

41. A method for detecting an otopathogen in a subject, comprising:
collecting a nasal sample from the subject;
combining a portion of the nasal sample with an extraction reagent to produce a test sample;
combining a portion of the test sample with an amplification reagent to produce an amplification sample comprising a portion of the test sample and reagents for amplification of otopathogen nucleic acid;
amplifying otopathogen nucleic acid, if present, by polymerase chain reaction (PCR) or by an isothermal amplification technique, in the amplification sample to produce an amplified sample; and
detecting amplified otopathogen nucleic acid, if present, in the amplified sample.

42. A method for identifying the etiology of acute otitis media in a clinically diagnosed subject, comprising:
collecting a nasal sample from the subject;
combining a portion of the nasal sample with an extraction reagent to produce a test sample;
combining a portion of the test sample with an amplification reagent to produce an amplification sample comprising a portion of the test sample and reagents for amplification of otopathogen nucleic acid;
amplifying otopathogen nucleic acid, if present, by an amplification technique in the amplification sample to produce an amplified sample; and
detecting amplified otopathogen nucleic acid, if present, in the amplified sample;
wherein detection of amplified otopathogen nucleic acid in the amplified sample confirms a clinical diagnosis of acute otitis media.

43. A method for detecting an otopathogen in a subject or for identifying the etiology of acute otitis media in a clinically diagnosed subject comprising:
collecting a sample from the subject;
optionally, combining a portion of the sample with a reagent to produce a test sample;
amplifying otopathogen nucleic acid, if present, by an amplification technique on the sample; and
detecting an otopathogen, if present, in the sample.

44. The method of claim 41 or claim 42, wherein the nasal sample is collected with a collection tool.

45. The method of claim 43, wherein the sample is a fluid sample from the subject.

46. The method of claim 45, wherein the fluid sample is sputum, oropharyngeal, saliva, middle or inner ear fluid, or a nasal sample.

47. The method of any one of claim 41, 42 or 46, wherein the nasal sample is a nasopharyngeal sample, an anterior naris sample, an inferior turbinate sample, a middle turbinate sample, or a superior turbinate sample.

48. The method of claim 47, wherein the nasal sample is collected with a swab.

49. The method of any one of claims 41-48, wherein the amplifying or detecting comprises multiplex PCR.

50. The method of any one of claims 41-49, wherein the amplifying comprises amplification of nucleic acid from H. influenzae, S. pneumoniae, or M. catarrhalis.

51. The method of any one of claims 41-50, wherein the amplifying comprises amplification of an antibiotic resistance gene from H. influenzae, S. pneumoniae, or M. catarrhalis.

52. The method of any one of claims 41-51, wherein the amplification reagent comprises a labeled probe unique for H. influenzae, S. pneumoniae, or M. catarrhalis nucleic acid.

53. The method of any one of claims 41-52, wherein the amplification reagent comprises a fluorescently labeled probe unique for H. influenzae, S. pneumoniae, or M. catarrhalis nucleic acid.

54. The method of claim 53, wherein the fluorescently labeled probe comprises a fluorophore and a quencher.

55. The method of claim 54, wherein amplification of H. influenzae, S. pneumoniae, or M. catarrhalis nucleic acid separates the fluorophore from the quencher.

56. The method of any of claims 41-55, wherein the detection comprises detection of a single nucleic acid, a single protein, or an amplified nucleic acid.

57. A method for treating acute otitis media in a subject, comprising:
obtaining a nasal sample from the subject;
optionally combining a portion of the nasal sample with an extraction reagent to produce a test sample;
combining a portion of the nasal sample or the test sample with an amplification reagent to produce an amplification sample comprising a portion of the test sample and reagents for amplification of otopathogen nucleic acid;
amplifying otopathogen nucleic acid, if present, in the amplification sample to produce an amplified sample; and
detecting amplified otopathogen nucleic acid, if present, in the amplified sample;
wherein, upon detection of amplified otopathogen nucleic acid, acute otitis media in the subject is treated with an antibiotic.

58. The method of claim 57, wherein said detecting provides a test result, wherein said test result is analyzed according to an algorithm described herein.

59. The method of claim 57 or claim 58, wherein the nasal sample is obtained with a collection tool.

60. The method of any one of claims 57-59, wherein the nasal sample is obtained with a swab.

61. The method of claim 60, wherein the nasal sample is a nasopharyngeal sample, an anterior naris sample, an inferior turbinate sample, a middle turbinate sample, or a superior turbinate sample.

62. The method of any one of claims 57-61, wherein the amplifying is by polymerase chain reaction (PCR) or by isothermal amplification.

63. The method of any one of claims 57-62, wherein the amplifying comprises amplification of nucleic acid from H. influenzae, S. pneumoniae, and M. catarrhalis.

64. The method of claim 63, wherein said detecting provides a test result, wherein said test result is analyzed according to an algorithm described herein.

65. The method of any one of claim 57-64, wherein the amplifying comprises amplification of an antibiotic resistance gene from H. influenzae, S. pneumoniae, or M. catarrhalis.

66. The method of any one of claims 57-65, wherein the amplification reagent comprises a labeled probe unique for a H. influenzae, S. pneumoniae, or M. catarrhalis nucleic acid.

67. The method of any one of claims 57-66, wherein the amplification reagent comprises a fluorescently labeled probe unique for H. influenzae, S. pneumoniae, or M. catarrhalis nucleic acid.

68. The method of claim 67, wherein the fluorescently labeled probe comprises a fluorophore and a quencher.

69. The method of claim 68, wherein amplification of H. influenzae, S. pneumoniae, or M. catarrhalis nucleic acid separates the fluorophore from the quencher.

70. The method of any of claims 57-69, wherein the detection of amplified otopathogen nucleic acid comprises detection of fluorescence.

71. The method of any one of claims 57-70, wherein the antibiotic selected for treatment of acute otitis media is based on effectiveness of the antibiotic against the detected otopathogen.

72. A method for treating acute otitis media in a subject, comprising:
obtaining a sample from the subject;
optionally combining a portion of the sample with a reagent to produce a test sample;
detecting otopathogen, if present, in the sample;
wherein, upon detection of otopathogen, acute otitis media in the subject is treated with an antibiotic or is recommended for no immediate antibiotic treatment.

73. The method of claim 72, wherein said detecting is via single molecule detection of a nucleic acid or a protein, or wherein said detecting is via detection of an amplicon.

74. The method of claim 72 or claim 73, further comprising wherein after upon detection of otopathogen, a decision algorithm as described herein is applied to determine treatment with an antibiotic that has or is not required to have beta-lactamase activity.

75. The method of any one of claims 72-74, wherein the sample is a fluid sample such as sputum, oropharyngeal, saliva, middle or inner ear fluid, or a nasal sample, or wherein the sample is a nasal sample such as a nasopharyngeal sample, an anterior naris sample, an inferior turbinate sample, a middle turbinate sample, or a superior turbinate sample.

76. A method comprising:
providing a test input to a diagnostic and treatment system, the test input including data on the presence of absence of a plurality of otopathogens, the otopathogens including a positive beta-lactamase H. influenzae, a negative beta-lactamase H. influenzae, S. pneumoniae, and M. catarrhalis in a patient sample; and
generating, via a treatment model implemented using a decision algorithm, at least one primary treatment protocol.

77. The method of claim 76, wherein the test input is a result of an assay capable of amplifying nucleic acid from the otopathogens, if present, in a patient sample.

78. The method of claim 76 or claim 77, wherein the patient sample is a nasal sample and the patient demonstrated symptoms of otitis media.

79. The method of any one of claims 76-78, wherein the decision algorithm comprises evaluating the test input to determine one or more of the following:
(i) if S. pneumonia, negative beta-lactamase H. influenzae, and positive beta-lactamase are absent and M. catarrhalis is present;
(ii) if S. pneumonia is absent and positive beta-lactamase H. influenzae and/or M. catarrhalis are present;
(ii′) if S. pneumonia negative beta-lactamase H. influenzae, positive beta-lactamase H. influenzae, and M. catarrhalis are absent,
(iii) if S. pneumonia is present and M. catarrhalis and/or positive beta-lactamase H. influenzae is/are present;
(iv) if S. pneumonia and negative beta-lactamase H. influenzae are present, and M. catarrhalis and positive beta-lactamase H. influenzae are absent; and
(v) if S. pneumonia, negative beta-lactamase H. influenzae, positive beta-lactamase H. influenzae, and M. catarrhalis are absent.

80. The method of claim 79, wherein
if (i), (ii) or (ii′) are true, the at least one primary treatment protocol includes providing no immediate antibiotic treatment;
if (iii) is true, the at least one primary treatment protocol includes providing immediate antibiotic treatment with an antibiotic with beta-lactamase activity; and/or
if (iv) is true, the at least one primary treatment protocol includes providing immediate antibiotic treatment with an antibiotic that does not require beta-lactamase activity.

81. The method of claim 80, wherein if (i) or (ii) are true, the at least one primary treatment protocol includes (x) observation with clinical follow-up and (y) providing a delayed prescription with appropriate clinical follow-up.

82. The method of claim 76, wherein the at least one primary treatment protocol is displayed on an output interface.

83. The method of any one of claims 76-82, further comprising providing a secondary treatment protocol.

84. The method of claim 83, wherein the second treatment protocol includes providing a rescue antibiotic treatment.

85. The method of claim 84, wherein the rescue antibiotic has beta-lactamase activity if positive beta-lactamase H. influenzae is present and/or M. catarrhalis is present.
